(19) United States
(12) Patent Application Publication
(10) Pub. No.: US 2022/0367004 A1
(43) Pub. Date: Nov. 17, 2022

(54) ASSESSING GUT HEALTH USING METAGENOME DATA

(71) Applicant: Mayo Foundation for Medical Education and Research, Rochester, MN (US)

(72) Inventors: Jaeyun Sung, Rochester, MN (US); Vinod K. Gupta, Rochester, MN (US)

(21) Appl. No.: 17/295,098

(22) PCT Filed: Nov. 20, 2020
(86) PCT No.: PCT/US2020/061557

§ 371 Date: May 19, 2021

Related U.S. Application Data
(60) Provisional application No. 62/938,827, filed on Nov. 21, 2019.

(51) Int. Cl. G16B 30/00; C12Q 1/6888; G16B 40/20; G16H 50/20

(52) U.S. Cl. CPC G16B 30/00; C12Q 1/6888; G16B 40/20; G16H 50/20; C12Q 1/6869

(57) ABSTRACT

Metagenome data can be obtained for a stool sample of the individual. An indication of presence of a microbial species in the stool sample of the individual can be determined based on the metagenome data for each microbial species of a pre-defined set of microbial species. Based on the indications of presence of the microbial species from the pre-defined set of microbial species, a relative presence of microbial species can be determined from a first pre-defined subset of the pre-defined set of microbial species to microbial species from a second pre-defined subset of the pre-defined set of microbial species. An assessment of the gut health of the individual can be provided based on the relative presence of microbial species in the stool sample from the first pre-defined subset to microbial species from the second pre-defined subset.

### CROSS-REFERENCE TO RELATED APPLICATIONS

This application claims the benefit of priority to U.S. Application Ser. No. 62/938,827, filed on Nov. 21, 2019. The content of U.S. Application Ser. No. 62/938,827 is considered part of the disclosure of the present document and is incorporated by reference in its entirety.

### BACKGROUND

Individuals working in this field of endeavor have investigated whether particular microbiomes in the human gut have an impact on triggering certain diseases.

### SUMMARY

This specification describes systems, methods, devices, and other techniques for assessing the health of an individual's gut microbiome based on the metagenome of a stool sample from the individual. The assessment, for example, can be provided in the form of a score (e.g., a Gut Microbiome Health Index (GMHI)) that portrays an overall health condition of the user's gut. Rather than portraying a link to specific diseases, the score or other forms of assessment can denote the degree to which a subject's sample portrays microbial taxonomic properties associated with overall health.

Implementations of the subject matter described herein include methods for assessing the gut health of an individual (e.g., a human or other mammal). The methods can include obtaining metagenome data for a stool sample of the individual. An indication of presence of the microbial species in the stool sample of the individual can be determined based on the metagenome data for each microbial species of a pre-defined set of microbial species. Based on the indications of presence of the microbial species from the pre-defined set of microbial species, a relative presence of microbial species can be determined from a first pre-defined subset of the pre-defined set of microbial species to microbial species from a second pre-defined subset of the pre-defined set of microbial species. An assessment of the gut health of the individual can be provided based on the relative presence of microbial species in the stool sample from the first pre-defined subset to microbial species from the second pre-defined subset.

Some implementations of the subject matter disclosed herein include a method for assessing the gut health of an individual. The method can include actions of obtaining metagenome data that describes the metagenome for a stool sample of the individual; determining, based on the metagenome data and for each microbial species of a pre-defined set of microbial species, an indication of presence of the microbial species in the stool sample of the individual; determining, based on the indications of presence of the microbial species from the pre-defined set of microbial species, a relative presence in the stool sample of microbial species from a first pre-defined subset of the pre-defined set of microbial species to microbial species from a second pre-defined subset of the pre-defined set of microbial species; and providing an assessment of the gut health of the individual based on the relative presence of microbial species in the stool sample from the first pre-defined subset to microbial species from the second pre-defined subset.

These and other implementations can further include one or more of the following features.

The indication of presence of the microbial species can include a binary indication that the microbial species either has a threshold level of abundance in the stool sample or does not have the threshold level of abundance in the stool sample.

The indication of presence of the microbial species can include an indication of a level of abundance of the microbial species in the stool sample.

The actions can further include obtaining the stool sample from the individual; and analyzing the stool sample to determine the metagenome data.

Analyzing the stool sample to determine the metagenome data for the stool sample can include performing at least one of a shotgun sequencing technique on the stool sample, a high-throughput sequencing technique on the stool sample, or a polymerase chain reaction (PCR) technique on the stool sample.

Microbial species in the pre-defined set of microbial species can be selected for inclusion in the pre-defined set based on having been determined to be a statistically significant indicator of gut health such that a presence or lack of presence of the microbial species in studied stool samples was statistically associated with either a healthy gut biome or an unhealthy gut biome.

The studied stool samples can each have been classified as being (i) associated with a healthy gut biome if the stool sample was obtained from an individual who was not identified as a having disease and who had a body mass index (BMI) within a normal range, or (ii) associated with an unhealthy gut biome if the stool sample was obtained from an individual who was identified as having disease or who had a BMI outside of the normal range.

The pre-defined set of microbial species can include fifty microbial species.

The first pre-defined subset of microbial species can consist of microbial species whose abundance in a stool sample is determined to be a statistically significant indicator of a healthy gut biome.

The second pre-defined subset of microbial species can consist of microbial species whose scarcity in studied stool samples is determined to be a statistically significant indicator of a healthy gut biome.

The first pre-defined subset of microbial species can consist of microbial species that are associated with healthy gut biomes.

The second pre-defined subset of microbial species can consist of microbial species that are associated with unhealthy gut biomes.

Determining the relative presence in the stool sample of microbial species from the first pre-defined subset to microbial species from the second pre-defined subset can include: determining a first aggregate indication of presence of microbial species from the first pre-defined subset; determining a second aggregate indication of presence of microbial species from the second pre-defined subset; and determining a relationship between the first aggregate indication of presence of microbial species from the first pre-defined subset to the second aggregate indication of presence of microbial species from the second pre-defined subset.

The relationship can include a ratio between the first aggregate indication of presence of microbial species from the first pre-defined subset to the second aggregate indication of presence of microbial species from the second pre-defined subset.

Providing the assessment of the gut health of the individual can include providing a score indicative of the relative presence in the stool sample of microbial species from the first pre-defined subset to microbial species from the second pre-defined subset.

The actions can further include normalizing the score such that a negative score indicates an unhealthy gut biome, a positive score indicates a healthy gut biome, and a zero score indicates a neutral gut biome.

The actions can further include comparing the score to a threshold value, and providing an indication of the gut health of the individual based on a result of the comparison of the score to the threshold value.

The actions can further include generating, based on at least one of the metagenome data or the assessment of the gut health of the individual, a behavioral recommendation that indicates a recommended behavior for the individual to improve gut health; and providing the behavior recommendation to the individual or another user.

The behavioral recommendation can include at least one of a dietary or fitness recommendation.

Generating the behavioral recommendation can include accessing, using a computing system, a model that stores data correlating various gut health assessments with corresponding behavioral recommendations.

Providing the behavioral recommendation can include at least one of presenting the behavioral recommendation on a screen of a computing device or transmitting a representation of the behavioral recommendation over a network.

Providing the assessment of the gut health of the individual can include presenting the assessment on a screen of a computing device.

The actions can further include using a machine-learning model to determine the relative presence in the stool sample of microbial species from the first pre-defined subset to the second pre-defined subset.

The machine-learning model can include an artificial neural network.

In some aspects, the methods described in this specification can be performed in whole or in part by a computing system. The computing system can include one or more computers in one or more locations configured to perform the actions of the methods described herein. In some implementations, the one or more computer-readable media are encoded with instructions that, when executed by one or more computers/processors, cause the computers/processors to perform actions of the methods described herein.

The details of one or more embodiments are set forth in the accompanying drawings and in the description below. Other features and advantages will be apparent from the description and drawings, and from the claims.

### BRIEF DESCRIPTION OF DRAWINGS

FIG. 1 is a flowchart depicting an example method for determining an assessment of an individual's overall gut health using metagenome data from a stool sample from the individual.

FIG. 2 is a flowchart depicting an example method for training and generating an aggregate gut health model.

FIG. 3 depicts an example system diagram for implementing the techniques described herein.

FIGS. 4A-4D depict integration of human stool metagenomes leads to a meta-dataset of healthy and non-healthy gut microbiomes.

FIGS. 5A-5B depict GMHI associated with high-density lipoprotein cholesterol (HDLC). (a) GMHI shows a moderately positive correlation with HDLC (Spearman's ρ=0.34, 95% CI: [0.28, 0.40], P=7.19×10−24), which is a key parameter of cardiovascular health, in 841 subjects. (b) Significantly higher abundances of HDLC were observed in subjects with positive GMHI compared to those with negative GMHI (two-sided Mann-Whitney U test, P=1.22×10−16). The sample size of each group, whose subjects' HDLC records were available in the original studies, is shown in parentheses. Standard box-and-whisker plots (e.g., center line, median; box limits, upper and lower quartiles; whiskers, 1.5 interquartile range; circles, outliers) are used to depict groups of numerical data.

FIGS. 6A-6H depict comparisons amongst GMHI and other ecological metrics in stratifying healthy from non-healthy phenotypes. (a-d) Significantly higher distributions of GMHI (P=5.06×10−212), Shannon diversity (P=8.50×10−9), and 80% abundance coverage (P=2.30×10−12) were observed in gut microbiomes of healthy than in those of non-healthy individuals, whereas higher species richness (P=2.30×10−46) was observed in non-healthy gut microbiomes. The strongest effect-size (Cliff's Delta, d) was seen with GMHI. (e-h) The healthy group was found to have a significantly higher distribution of GMHIs than all but one (SA) of the twelve non-healthy phenotypes. For Shannon diversity and 80% abundance coverage, only three non-healthy phenotypes (CD, OB, and T2D) were found to have significantly different distributions compared to healthy; both properties were higher in healthy than in CD, OB, and T2D. For species richness, seven (ACVD, CA, CC, OB, OW, RA, and T2D) of the twelve non-healthy phenotypes were observed to have significantly higher richness than healthy; in contrast, only CD showed significantly lower richness compared to healthy. All P-values shown above the violin plots were found using the two-sided Mann-Whitney U test. *, P<0.001 in two-sided Mann-Whitney U test; ns, not significant. The sample size of each group is shown in parentheses. ACVD, atherosclerotic cardiovascular disease; CA, colorectal adenoma; CC, colorectal cancer; CD, Crohn's disease; IGT, impaired glucose tolerance; OB, obesity; OW, overweight; RA, rheumatoid arthritis; SA, symptomatic atherosclerosis; T2D, type 2 diabetes; UC, ulcerative colitis; and UW, underweight. Standard box-and-whisker plots (e.g., center line, median; box limits, upper and lower quartiles; whiskers, 1.5 interquartile range; circles, outliers) are used to depict groups of numerical data.

FIGS. 7A-7B depict proportions and Shannon diversity with respect to GMHI. (a) All 4,347 metagenomes were binned according to their GMHI values (x-axis). Each gray bar indicates the total number of samples in each bin (y-axis, right). Points indicate proportions (i.e., percentages) of samples in each bin corresponding to either healthy or non-healthy individuals (y-axis, left). In bins with a positive range of GMHIs, the majority of samples classified as healthy; in contrast, samples in bins with a negative range of GMHIs mostly classified as non-healthy. This trend was more pronounced towards bins on the far right and left. (b) GMHI stratifies healthy (n=2,636) and non-healthy (n=1,711) groups more strongly compared to Shannon diversity. Each point in the scatter-plot corresponds to a metagenome sample (4,347 in total). Histograms show the distribution of healthy and non-healthy samples based on the parameter of each axis. In general, GMHI and Shannon diversity demonstrate a weak correlation (Spearman's ρ=0.17, 95% CI: [0.14, 0.19], P=1.7×10−28). The P-value (HO: ρ=0) was determined by using a t-distribution with n-2 degrees of freedom, where n is the total number of observations.

FIGS. 8A-8D depict indications of GMHI generally outperforming other microbiome ecological characteristics in distinguishing case and control across multiple study-specific comparisons. In each of the twelve studies wherein at least ten case (i.e., disease or abnormal bodyweight conditions) and at least ten control (i.e., healthy) subjects were available, stool metagenomes were analyzed to compare (a) GMHI, (b) Shannon diversity, (c) 80% abundance coverage, and (d) species richness between healthy and non-healthy phenotype(s). GMHI was found to have a significantly higher distribution in healthy for eleven case-control comparisons across nine different studies; Shannon diversity and 80% abundance coverage were found to have significantly higher distributions in healthy for two and four case-control comparisons (across two and four studies), respectively; and species richness was found to have a significantly lower distributions in healthy for three case-control comparisons across three different studies. Each study's phenotype sample size is shown in parentheses to the right of the phenotype abbreviation. Standard box-and-whisker plots (e.g., center line, median; box limits, upper and lower quartiles; whiskers, 1.5 interquartile range; points, samples) are used to depict groups of numerical data. The same colors in box-plots were used for the same phenotypes. P-values (two-sided Mann-Whitney U test) for each study-specific comparison between healthy and non-healthy phenotypes are shown adjacent to the boxplots accordingly: * and L indicates significantly different distributions consistent with, and opposite to, respectively, the previously observed results when healthy and non-healthy groups were compared in aggregate. * or ψ, 0.01≤P-value<0.05; ** or ψψ, 0.001≤P-value<0.01; *** or ψψψ, 0.0001≤P-value<0.001; **** or ψψψψ, P-value<0.0001. ACVD, atherosclerotic cardiovascular disease; CA, colorectal adenoma; CC, colorectal cancer; CD, Crohn's disease; IGT, impaired glucose tolerance; OB, obesity; OW, overweight; RA, rheumatoid arthritis; SA, symptomatic atherosclerosis; T2D, type 2 diabetes; UC, ulcerative colitis; and UW, underweight.

FIGS. 9A-9B are illustrations showing how GMHI demonstrates strong reproducibility on an independent validation cohort. The validation cohort (679 stool metagenome samples) consisted of twelve total sub-cohorts ranging across eight healthy and non-healthy phenotypes from nine different studies. (a) GMHIs from stool metagenomes of the healthy group were significantly higher than those of the non-healthy group (two-sided Mann-Whitney U test, P=3.49×10−28). d, Cliff's Delta. (b) All three healthy sub-cohorts (H1, H2, and H3) were found to have significantly higher distributions of GMHI than seven (of nine) non-healthy sub-cohorts (AS4, CC5-I, CC5-J, CD6, LC7, NAFLD8, and RA9). No significant differences were found amongst H1, H2, and H3. The number in superscript adjacent to phenotype abbreviations corresponds to a particular study used in validation. Standard box-and-whisker plots (e.g., center line, median; box limits, upper and lower quartiles; whiskers, 1.5 interquartile range; points, samples) are used to depict groups of numerical data. * indicates significantly higher distribution in healthy sub-cohort (two-sided Mann-Whitney U test, P<0.01). The number adjacent to * indicates the healthy sub-cohort (H1, H2, or H3) to which the respective sub-cohort was compared. The sample size of each group or cohort is shown in parentheses. AS, ankylosing spondylitis; CA, colorectal adenoma; CC, colorectal cancer; CD, Crohn's disease, H, healthy; LC liver cirrhosis; NAFLD, non-alcoholic fatty liver disease; RA, rheumatoid arthritis.

FIG. 10 shows an example of a computing device and a mobile computing device that can be used to implement the techniques described herein.

FIG. 11 is a table of various characteristics of human stool metagenome datasets analyzed in the study.

FIG. 12 is a table indicating describing sets of microbial species.

### DETAILED DESCRIPTION

This specification describes systems, methods, devices, and other techniques for assessing the health of an individual's gut microbiome based on the metagenome of a stool sample from the individual. The assessment, for example, can be provided in the form of a score (e.g., a Gut Microbiome Health Index (GMHI)) that portrays an overall health condition of the user's gut microbiome. Rather than portraying a link to specific diseases, the score or other forms of assessment can denote the degree to which a subject's stool sample portrays microbial taxonomic properties associated with overall health of the individual.

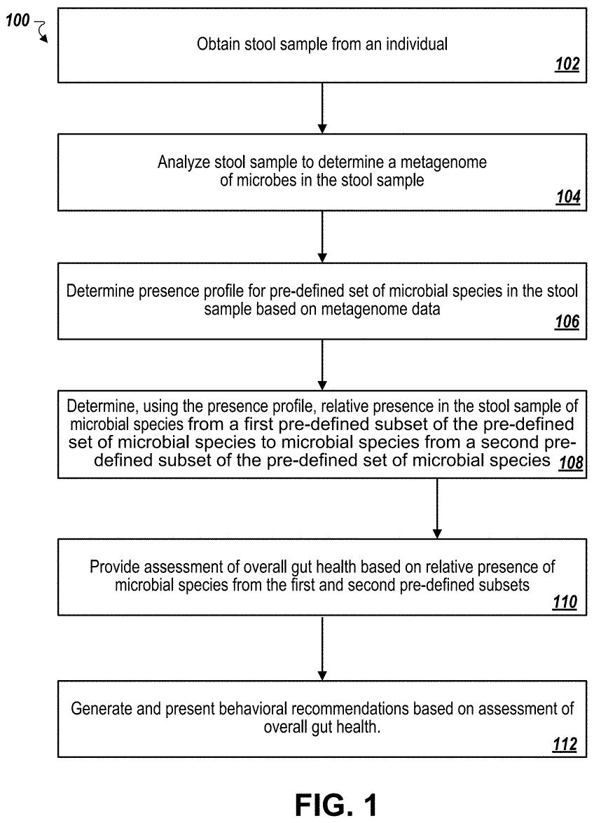

Referring to FIG. 1, a flowchart is depicted of an example method 100 for assessing an overall gut health of an individual. The method includes actions at stages 102-112. The actions in stages 104-112 can be performed in whole or in part by a computing system.

A stool sample of an individual is obtained (102). The stool sample is then analyzed to determine a metagenome of microbes in the stool sample (104). The analysis can be performed, for example, using shotgun sequencing, high-throughput sequencing, polymerase chain reaction (PCR), or a combination of these or other suitable sequencing techniques. In some implementations, the metagenome is formatted into data that can be processed by a computing system to provide an assessment of the individual's gut health.

Next, based on the metagenome data for the stool sample, a presence profile is determined for a pre-defined set of microbial species with respect to the stool sample (106). The presence profile can indicate, for each microbial species in the pre-defined set, information about the presence of the microbial species in the stool sample. For example, the profile can indicate whether a given species was or was not detected as being present in the stool sample, whether a given species was or was not detected as having at least a threshold abundance in the stool sample, a level of abundance of a given species in the stool sample, or a combination of two or more of these. In some implementations, the pre-defined set of species is a limited set of microbial species that has been determined through empirical analysis to be highly indicative of an overall health condition of an individual's gut microbiome. For example, the abundance of certain microbial species in the pre-defined set may be highly correlated with a healthy gut, whereas the abundance of other microbial species in the pre-defined set may be highly correlated with an unhealthy gut biome. As another example, the scarcity of certain microbial species in the pre-defined set may be highly correlated with a healthy gut, whereas the scarcity of other microbial species in the pre-defined set may be highly correlated with an unhealthy gut biome. In the example study implementation described below, fifty microbial species were identified as being associated with overall health; 7 and 43 of which were abundant and scarce, respectively, in the healthy cohort compared to the unhealthy one.

The method can then include determining, based on the presence profile, a relative presence in the stool sample of microbial species from a first pre-defined subset of the pre-defined set of microbial species to microbial species from a second pre-defined subset of the pre-defined set of microbial species (108). In some implementations, a ratio is determined of the aggregate presence of a subset of microbial species associated with a “healthy” condition relative to the aggregate presence of a subset of microbial species associated with an “unhealthy” condition of the individual. A higher ratio of “healthy” species to unhealthy species in the stool sample can indicate a greater likelihood of a higher health level, while a higher ratio of “unhealthy” species to healthy species in the stool sample can indicate a greater likelihood of a lower overall health level. The method can then provide to a user (e.g., to the individual whose stool sample was obtained or to a healthcare provider), an assessment of the overall gut health of the individual based on the relative presence of microbial species from the first and second pre-defined subsets (110). In some implementations, the assessment is provided in the form of a score (e.g., a Gut Microbiome Health Index (GMHI)) that is normalized so that a GMHI index of zero indicates an “average” or “neutral” gut health due to a balance of microbial species associated with both healthy and unhealthy conditions; a positive GMHI index indicates an overall healthy condition of the individual's gut; and a negative GMHI index indicates an overall unhealthy condition of the individual's gut. In some implementations, the system can then generate and present behavioral recommendations based on the individual's gut health assessment (112). For example, a GMHI index of zero or a negative value can indicate a lack of (or deficiency in) “healthy” species needed to maintain an individual overall gut health. The system (or a healthcare professional) can then recommend the following dietary and/or other behavioral recommendations to a user: 1) Consume more of the “healthy” microbes directly via supplement probiotics and fermented foods, which are natural source of probiotics; 2) Include prebiotics or fiber-rich foods in the diet, for instance, fermented vegetables, kefir, kimchi, kombucha, miso, sauerkraut, raw dandelion greens, leeks, onions, garlic, asparagus, whole wheat, spinach, beans, bananas, and tempeh. Prebiotics are usually undigestible carbohydrates, which feed the beneficial healthy bacteria in the colon; 3) Reduce the consumption of high fat and high sugar foods, and artificial sweeteners; 4) Modify behavior towards reducing stress, have regular exercise, have enough sleep, and engage in meditation that can help reduce stress levels; 5) Avoid unnecessary use of antibiotics, which have been found to damage the gut flora; and/or 6) Reduce consumption of animal products and increase plant-based products in diet. In some implementations, a computer system may store 1) data representing GMHI scores/indices (or ranges of GMHI scores/indices), 2) data representing dietary/behavioral recommendations, and 3) data correlating all or some of the GMHI scores/indices (or ranges of GMHI scores/indices) with one or more dietary and/or behavioral recommendations. Thus, when the system determines or obtains an indication of a GMHI index for an individual, it may access the stored data to lookup one or more dietary and/or behavioral recommendations corresponding to the GMHI index, and may present the GMHI index to the individual or another user. For example, the recommendation may be presented in an alert or notification to the user, may be formatted and presented in a webpage or native application interface on a user device (e.g., a smartphone, tablet, notebook, or desktop computer), may be sent in a text message (e.g., SMS message) to the individual or other user, and/or may be sent in an electronic mail message to the individual or other user.

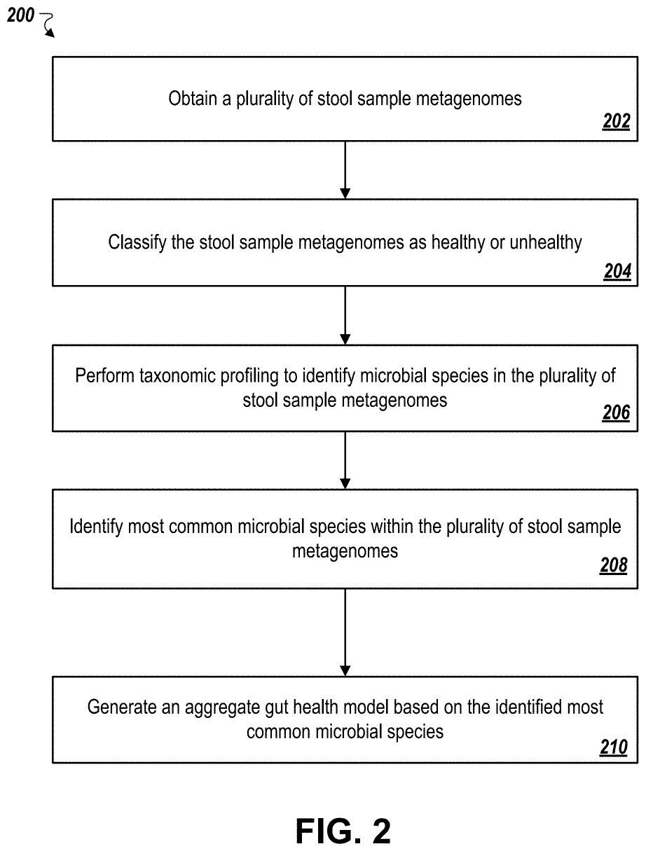

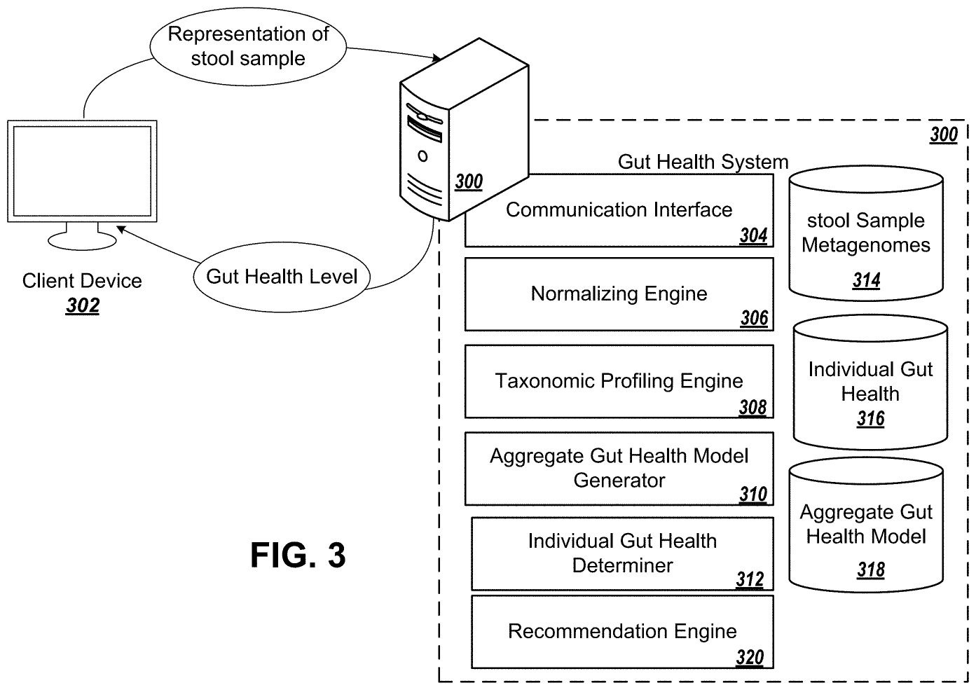

FIG. 2 depicts an example method 200 for training and generating an aggregate gut health model. At stage 202, a set of stool sample metagenomes are obtained. In some implementations, the obtained samples can include stool samples of individuals. At stage 204, each of the obtained stool sample metagenomes can be classified as either healthy or unhealthy. Classifying the stool samples can include analyzing a body mass index (BMI) associated with an individual whose stool sample was obtained. For example, if a BMI associated with a stool sample falls under a range of underweight (BMI<18.5), overweight (BMI≥25 & <30), or obesity (BMI≥30), that stool sample is classified as unhealthy. Additionally, if the individual was known to have certain diseases, the stool sample can be classified as unhealthy. In contrast, if the BMI falls within a normal range and the individual is not known to have certain diseases, then the stool sample can be classified as healthy. At stage 206, the method performs taxonomic profiling to identify microbial species in the plurality of stool sample metagenomes. A number of microbial species present in each of the plurality of stool samples can be identified. In some implementations, metagenomics reads can be classified to taxonomies in order to identify a number of bacteria, archaea, viruses, and/or eukaryotes that are identified from the plurality of stool sample metagenomes.

Next, at stage 208, the most common (e.g., most relevantly abundant) microbial species within the plurality of stool sample metagenomes can be identified. Microbial species abundance profiles can be generated for each stool sample in the set of stool samples to identify the most prevalent microbial species in each stool sample, such as the smallest subset of microbial species that provide at least a specified threshold (e.g., 80%) of the total relative abundance.

Once the most common microbial species within the plurality of stool samples are identified, an aggregate gut health model can be generated at stage 210. In some implementations, the model can be generated using machine learning techniques. The model can be implemented on a computing system and configured to output an overall assessment of gut health based on the metagenome of a stool sample. The overall assessment can be in the form of a score such as a Gut Microbiome Health Index (GMHI). The GMHI can represent, in a single quantitative measure, an accumulation of multi-dimensional information reflective, for example, of a count of microbial species observed to be present in the stool sample, their relative abundances, and their taxonomic diversity in the sample. The GMHI can denote a degree to which an individual's stool sample metagenome portrays microbial taxonomic properties associated with health of the individual's gut. A positive GMHI can allow the individual's stool sample to be classified as healthy while a negative GMHI allows the individual's stool sample to be classified as unhealthy. In some implementations, the classification can be different, including but not limited to a healthy classification being a number and/or other value above a certain predetermined threshold and an unhealthy classification being a number and/or value below a certain predetermined threshold. Moreover, in some implementations, a GMHI that is equal to 0 or some other predetermined neutral value can indicate that the individual's gut has an equal balance of healthy and unhealthy microbial species.

FIG. 2 depicts an example system diagram for implementing aspects of the techniques described herein. A client device 202 can be in communication with a gut health server system 200. The server 200 can be local, connected to the client device 202, and/or a remote server. The client device 202 and the server 200 can communicate wirelessly (e.g., BLUETOOTH, WIFI) and/or through a wired connection (e.g., ETHERNET). The client device 202 can be a computing device, such as a laptop, desktop, workstation, and/or a mobile computing device, such as a smartphone or other similar computing devices.

The client device 202 can be configured to provide inputs to the server 200, including, for example, data representative of metagenomes for one or more stool samples of an individual. The metagenome data can be inputted into the client device 202 by a healthcare provider and/or any other professional (e.g., lab specialist/analyst) who handles/has access to the stool metagenome samples. The server 200 can be configured to perform analysis of the metagenome data and return to the client device 202 an assessment of the gut health level of the individual from which each stool sample was obtained.

As depicted, the server 200 can comprise a communication interface 204, a normalizing engine 206, a taxonomic profiling engine 208, an aggregate gut health model generator 210, an individual gut health determiner 212, a recommendation engine 220, a stool sample metagenomes database 214, an individual gut health database 216, and an aggregate gut health model 218. The communication interface 204 can be configured to allow the server 200 to communicate with the client device 202, as previously discussed. When the server 200 receives input of the representations of stool samples (e.g., genetic sequencing data) from the client device 202, the server 200 can store those representations in the stool sample metagenomes database 214. The normalizing engine 206 can access the representations stored in the database 214 to classify each of the representations as health or unhealthy (refer to FIG. 1, step 104) based on metadata about the health condition of the individual from whom the corresponding stool sample was obtained (e.g., BMI, disease state). The taxonomic profiling engine 208 can then be configured to take the normalized (e.g., classified) representations from the normalizing engine 206 and perform taxonomic profiling to identify a presence or lack of presence of various microbial species in the representations of the stool samples (refer to FIG. 1, step 106). The engine 208 can further be configured to identify the most common microbial species within the representations of the stool samples (refer to FIG. 1, step 108). Then, the aggregate gut health model generator 210 can generate the aggregate gut health model (e.g., GMHI) based on the identified most common microbial species (refer to FIG. 1, step 110). Once the model is generated, the model can be stored in the aggregate gut health model 218.

The individual gut health determiner 212 can then be configured to coordinate activities in which, for example, it feeds inputs into the model 218 to generate outputs from the model 218. In other words, an individual representation of a stool sample (e.g., metagenome data for the stool sample) can be received from the client device 202 which the individual gut health determiner 212 can analyze using model 218 to determine an assessment (e.g., GMHI) of the gut health of the individual according to the process described with respect to FIG. 1. In this manner, system 200 can output to the client device 202 a gut health level of the individual whose representation of a stool sample was received by the server 200. Further, the recommendation engine 220 can be configured to access the indication of the overall gut health of the individual from the individual gut health database 216 and generate a behavioral recommendation or suggestion to improve the overall gut health of the individual. The suggestion generated by the recommendation engine 220 can also be outputted to the client device 202 along with a gut health level of the individual. In some implementations, the suggestion(s) can be stored with the indication of the overall gut health of the individual in the individual gut health database 218.

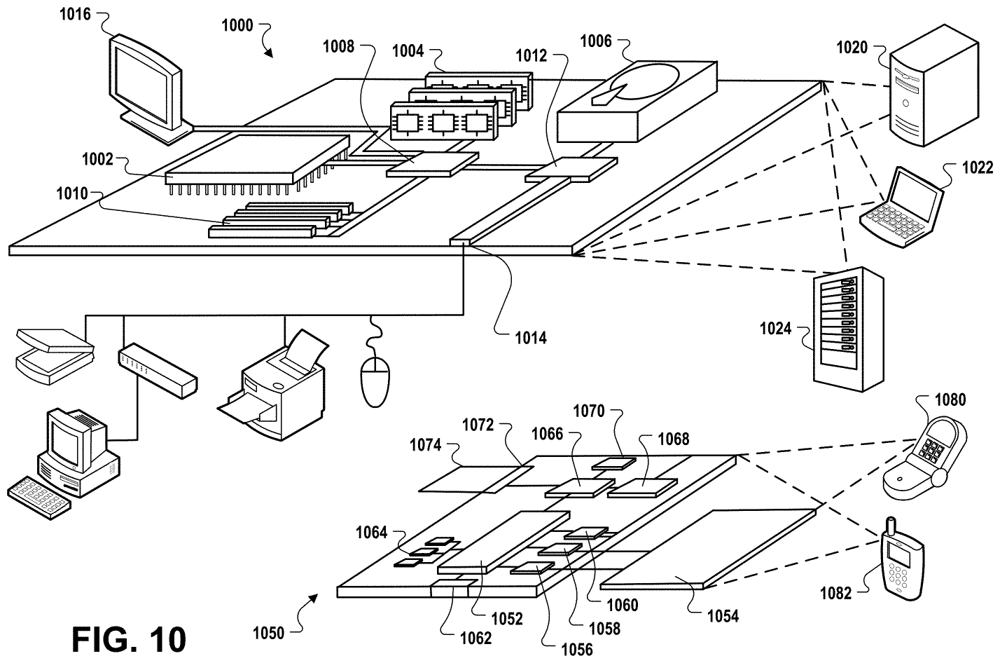

FIG. 10 shows an example of a computing device 1000 and a mobile computing device that can be used to implement computer-based embodiments of the techniques described herein. The computing device 1000 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1000 includes a processor 1002, a memory 1004, a storage device 1006, a high-speed interface 1008 connecting to the memory 1004 and multiple high-speed expansion ports 1010, and a low-speed interface 1012 connecting to a low-speed expansion port 1014 and the storage device 1006. Each of the processor 1002, the memory 1004, the storage device 1006, the high-speed interface 1008, the high-speed expansion ports 1010, and the low-speed interface 1012, are interconnected using various busses, and may be mounted on a common motherboard or in other manners as appropriate. The processor 1002 can process instructions for execution within the computing device 1000, including instructions stored in the memory 1004 or on the storage device 1006 to display graphical information fora GUI on an external input/output device, such as a display 1016 coupled to the high-speed interface 1008. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 1004 stores information within the computing device 1000. In some implementations, the memory 1004 is a volatile memory unit or units. In some implementations, the memory 1004 is a non-volatile memory unit or units. The memory 1004 may also be another form of computer-readable medium, such as a magnetic or optical disk.

The storage device 1006 is capable of providing mass storage for the computing device 1000. In some implementations, the storage device 1006 may be or contain a computer-readable medium, such as a floppy disk device, a hard disk device, an optical disk device, or a tape device, a flash memory or other similar solid state memory device, or an array of devices, including devices in a storage area network or other configurations. The computer program product may also contain instructions that, when executed, perform one or more methods, such as those described above. The computer program product can also be tangibly embodied in a computer- or machine-readable medium, such as the memory 1004, the storage device 1006, or memory on the processor 1002.

The high-speed interface 1008 manages bandwidth-intensive operations for the computing device 1000, while the low-speed interface 1012 manages lower bandwidth-intensive operations. Such allocation of functions is exemplary only. In some implementations, the high-speed interface 1008 is coupled to the memory 1004, the display 1016 (e.g., through a graphics processor or accelerator), and to the high-speed expansion ports 1010, which may accept various expansion cards (not shown). In the implementation, the low-speed interface 1012 is coupled to the storage device 1006 and the low-speed expansion port 1014. The low-speed expansion port 1014, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 1000 may be implemented in a number of different forms, as shown in the figure. For example, it may be implemented as a standard server 1020, or multiple times in a group of such servers. In addition, it may be implemented in a personal computer such as a laptop computer 1022. It may also be implemented as part of a rack server system 1024. Alternatively, components from the computing device 1000 may be combined with other components in a mobile device (not shown), such as a mobile computing device 1050. Each of such devices may contain one or more of the computing device 1000 and the mobile computing device 1050, and an entire system may be made up of multiple computing devices communicating with each other.

The mobile computing device 1050 includes a processor 1052, a memory 1064, an input/output device such as a display 1054, a communication interface 1066, and a transceiver 1068, among other components. The mobile computing device 1050 may also be provided with a storage device, such as a micro-drive or other device, to provide additional storage. Each of the processor 1052, the memory 1064, the display 1054, the communication interface 1066, and the transceiver 1068, are interconnected using various buses, and several of the components may be mounted on a common motherboard or in other manners as appropriate.

The processor 1052 can execute instructions within the mobile computing device 1050, including instructions stored in the memory 1064. The processor 1052 may be implemented as a chipset of chips that include separate and multiple analog and digital processors. The processor 1052 may provide, for example, for coordination of the other components of the mobile computing device 1050, such as control of user interfaces, applications run by the mobile computing device 1050, and wireless communication by the mobile computing device 1050.

The processor 1052 may communicate with a user through a control interface 1058 and a display interface 1056 coupled to the display 1054. The display 1054 may be, for example, a TFT (Thin-Film-Transistor Liquid Crystal Display) display or an OLED (Organic Light Emitting Diode) display, or other appropriate display technology. The display interface 1056 may comprise appropriate circuitry for driving the display 1054 to present graphical and other information to a user. The control interface 1058 may receive commands from a user and convert them for submission to the processor 1052. In addition, an external interface 1062 may provide communication with the processor 1052, so as to enable near area communication of the mobile computing device 1050 with other devices. The external interface 1062 may provide, for example, for wired communication in some implementations, or for wireless communication in other implementations, and multiple interfaces may also be used.

The memory 1064 stores information within the mobile computing device 1050. The memory 1064 can be implemented as one or more of a computer-readable medium or media, a volatile memory unit or units, or a non-volatile memory unit or units. An expansion memory 1074 may also be provided and connected to the mobile computing device 1050 through an expansion interface 1072, which may include, for example, a SIMM (Single In Line Memory Module) card interface. The expansion memory 1074 may provide extra storage space for the mobile computing device 1050, or may also store applications or other information for the mobile computing device 1050. Specifically, the expansion memory 1074 may include instructions to carry out or supplement the processes described above, and may include secure information also. Thus, for example, the expansion memory 1074 may be provide as a security module for the mobile computing device 1050, and may be programmed with instructions that permit secure use of the mobile computing device 1050. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. The computer program product contains instructions that, when executed, perform one or more methods, such as those described above. The computer program product can be a computer- or machine-readable medium, such as the memory 1064, the expansion memory 1074, or memory on the processor 1052. In some implementations, the computer program product can be received in a propagated signal, for example, over the transceiver 1068 or the external interface 1062.

The mobile computing device 1050 may communicate wirelessly through the communication interface 1066, which may include digital signal processing circuitry where necessary. The communication interface 1066 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1068 using a radio-frequency. In addition, short-range communication may occur, such as using a Bluetooth, WiFi, or other such transceiver (not shown). In addition, a GPS (Global Positioning System) receiver module 1070 may provide additional navigation- and location-related wireless data to the mobile computing device 1050, which may be used as appropriate by applications running on the mobile computing device 1050.

The mobile computing device 1050 may also communicate audibly using an audio codec 1060, which may receive spoken information from a user and convert it to usable digital information. The audio codec 1060 may likewise generate audible sound for a user, such as through a speaker, e.g., in a handset of the mobile computing device 1050. Such sound may include sound from voice telephone calls, may include recorded sound (e.g., voice messages, music files, etc.) and may also include sound generated by applications operating on the mobile computing device 1050.

The mobile computing device 1050 may be implemented in a number of different forms, as shown in the figure. For example, it may be implemented as a cellular telephone 1080. It may also be implemented as part of a smart-phone 1082, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In situations in which the systems, methods, devices, and other techniques here collect personal information (e.g., context data) about users, or may make use of personal information, the users may be provided with an opportunity to control whether programs or features collect user information (e.g., information about a user's social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

Example Implementation Study

In this section, a study is described that involved an example implementation of the disclosed techniques for identifying a microbial taxonomic signature associated with gut wellness.

Discussion

A growing body of evidence has linked alterations in the gut microbiome to major illnesses. Microbiome data is highly complex with enormous sample-level heterogeneity. As such, one object of certain implementations of the disclosed techniques is to provide a simple measure to quantify the degree of wellness, or the divergence away from a healthy condition of an individual (e.g., a patient, person, or other individual).

The example study described here seeks to address this challenge by integrating massive amounts of publicly available data (>4,300 publicly-available, shotgun metagenomic data of gut microbiomes). The study identified a small consortium of 50 microbial species associated with human health, 7 and 43 of which were abundant and scarce, respectively, in the healthy cohort compared to the unhealthy one. The study developed the GMHI for determining the health or dysbiotic status of a gut microbiome based on a stool specimen. GMHI is a biologically interpretable, quantitative metric formulated based on the relative abundances of the aforementioned microbial species, and can be applied to population-wide microbiome datasets. This framework can also be applied to other niches of the human body, e.g., quantifying health in skin or oral microbiomes. On independent validation datasets, this example study demonstrated the potential of GMHI to distinguish between health and disease, showing strong prediction results for healthy individuals, and cohorts with auto-immune disorders and liver disease.

Several limitations of the example study should be noted when interpreting the results. First, as the stool metagenome samples were collected from over 40 independent studies, the study cannot entirely exclude the possibility of experimental and technical inter-study batch effects (as is the case for any meta-analysis). The study's efforts to curtail these batch effects include: i) consensus preprocessing, i.e. for all samples, downloading raw metagenomes (e.g., .fastq files) and re-processing them uniformly using identical computational methods; and ii) rather than comparing averages between populations, using frequencies of a signal (in the form of sample coverage of ‘present’ microbes) as a measure to identify significantly associated microbial features. Second, the example study does not include all publicly available stool shotgun metagenomes studies and samples due to the study's strict selection criteria and reasoning. Certainly, more studies and/or samples can be used to take into consideration even more sources of heterogeneity. Third, the study's metagenomic analyses were limited to species-level taxonomies, although microbial strains are the clinically informative and actionable unit. Moreover, different strains within the same species can have different associations with health or disease, which may not be captured in the example study. Fourth, for the example study's unhealthy cohort, samples from only twelve disease or abnormal body weight conditions were pooled. In some implementations, more pathological states may be linked to the gut microbiome, including neurodegenerative and psychiatric diseases that were not included in the study's consensus metagenomic dataset. And lastly, the example study did not consider functional profiles to define gut ecosystem health, as this was outside the scope of the study.

Methods

Multi-study integration of human stool metagenomes. Keyword searches (e.g., “gut microbiome”, “metagenome”, “whole genome shotgun”) were performed in PubMed and Google Scholar for published studies with publicly available whole-genome shotgun (WGS) metagenome data of human stool (gut microbiome) and corresponding subject meta-data (as of March 2018). In studies where multiple samples were taken per individual across different time-points, only the first or baseline sample in the original study was included. Studies were excluded pertaining to diet or medication interventions, or those with fewer than 10 samples. Samples from subjects who were less than 10 years of age were also excluded from the analysis. Lastly, samples that were collected from disease controls, but were not reported as healthy nor had any mentioning of diagnosed disease in the original study, were excluded from our analysis. Raw sequence files (.fastq) were downloaded from the NCBI Sequence Read Archive (SRA) and European Nucleotide Archive (ENA) databases for the study analysis.

Re-classification of healthy samples based on reported BMI. Healthy individuals, regardless of whether they had been determined as healthy in the original studies, were considered to be part of the non-healthy group if their reported BMI fell within the range of underweight (BMI<18.5), overweight (BMI≥25 & <30), or obese (BMI≥30). Stool metagenome samples from such individuals were re-classified as underweight, overweight, or obese in the analysis.

Quality control of sequenced reads. Sequence reads were processed with the KneadData v0.5.1 quality-control pipeline, which uses Trimmomatic v0.36 and Bowtie2 v0.1 for removal of low-quality read bases and human reads, respectively. Trimmomatic v0.36 was run with parameters SLIDINGWINDOW:4:30, and Phred quality scores were thresholded at ‘<30’. Illumina adapter sequences were removed, and trimmed non-human reads shorter than 60 bp in nucleotide length were discarded. Potential human contamination was filtered by removing reads that aligned to the human genome (reference genome hg19). Furthermore, stool metagenome samples of low read count after quality filtration (<1M reads) were excluded from our analysis.

Species-level taxonomic profiling. Taxonomic profiling was done using the MetaPhIAn2 v2.7.0 phylogenetic clade identification pipeline³² using default parameters. Briefly, MetaPhIAn2 classifies metagenomic reads to taxonomies based on a database (mpa_v20_m200) of clade-specific marker genes derived from ˜17,000 microbial genomes (corresponding to ˜13,500 bacterial and archaeal, ˜3,500 viral, and ˜110 eukaryotic species).

Sample-filtering based on taxonomic profiles. After taxonomic profiling, the following stool metagenome samples were discarded from the analysis: i) samples composed of more than 5% unclassified taxonomies (100 samples); and ii) phenotypic outliers according to a dissimilarity measure. More specifically, Bray-Curtis distances were calculated between each sample of a particular phenotype and a hypothetical sample in which the species' abundances were taken from the medians across those samples. A sample was considered as an outlier, and thereby removed from further analysis, when its dissimilarity exceeded the upper and inner fence (i.e., >1.5 times outside of the interquartile range above the upper quartile and below the lower quartile) amongst all dissimilarities. This process removed 67 metagenome samples.

Species-removal based on taxonomic profiles. As taxonomic assignment based on clade-specific marker genes may be problematic for viruses, this study excluded the 298 of viral origin from analysis. Species that were labeled as either unclassified or unknown (118 species), or those of low prevalence (i.e., observed in <1% of the samples included in our meta-dataset; 472 species), were also excluded. Eventually, 313 microbial species across 4,347 stool metagenome samples remained in the study for further analysis.

Principal Coordinate Analysis based on taxonomic profiles. The R packages ‘ade4’ v1.7-15 and ‘vegan’ v2.5.6 were used to perform Principal Coordinate Analysis (PCoA) ordination with Bray-Curtis dissimilarity as the distance measure on the stool metagenome samples, which were comprised of arcsine square root transformed relative abundances of the aforementioned 313 microbial species identified by MetaPhIAn2. 999 permutations (‘adonis2’ function in the R ‘vegan’ package v2.5.6) were performed, while random permutations were constrained within studies by using the ‘strata’ option.

Calculation of microbiome ecological characteristics. The R package ‘vegan’ v2.5.6 was used to calculate Shannon diversity (Shannon index) and species richness based on the species abundance profiles for each sample of our meta-dataset. To identify the 80% abundance coverage for a stool metagenome sample, the smallest number of microbial species that comprise at least 80% of the total relative abundance was identified.

Identifying microbial species more frequently observed in Healthy than in Non-healthy (and vice versa).
a) Let p_{H,m} and p_{N,m} be the prevalence of microbial species m, i.e., proportion of samples in a given group where m is ‘present’ (or relative abundance ≥1.0×10⁻⁵), in the healthy group H and non-healthy group N, respectively. Remark: The relative abundances for all detectable species in a microbiome (metagenome) sample sums to 1.
b) For m, the prevalence fold-change fₘ^{H,N} and prevalence difference dₘ^{H,N}, defined as

$$\frac{{p}_{H , m}}{{p}_{N , m}}$$

and p_{H,m}-p_{N,m}, respectively, is identified.
c) Let θ_{f} and θ_{d} be defined as the minimum thresholds for fₘ^{H,N} and dₘ^{H,N}, respectively. For all detectable species in a microbiome sample, those that satisfy fₘ^{H,N}≥θ_{f} and dₘ^{H,N}≥θ_{d} are identified. These species are included as an element of ‘Health-prevalent’ species M_{H}, or the set of species more frequently observed in group H than in group N.
d) To identify ‘Health-scarce’ species M_{N}, or the set of species more frequently observed in group N than in group H, steps b) through c) are repeated with the following considerations:
   i. For m, let fₘ^{N,H} and dₘ^{N,H} be defined as pN,m/pH,m and p_{N,m}-p_{H,m}, respectively.
   ii. The same thresholds θ_{f} and θ_{d} are used to identify M_{N}. In this regard, the species that are eventually chosen to compose M_{H} and M_{N} are both dependent on θ_{f} and θ_{d}.
   iii. Finally, all detectable species that satisfy fₘ^{N,H}≥θ_{f} and dₘ^{N,H}≥θ_{d} are included in M_{N}.

Identifying ψ_{M}_{_{H}} (or ψ_{M}_{_{N}}), i.e., the ‘collective abundance’ of species in M_{H} (or M_{N}) in a microbiome sample.
a) ψ_{M}_{_{H}} is defined as the ‘collective abundance’ of M_{H} species in a microbiome sample. The calculation of ψ_{M}_{_{H}} takes into consideration the following:
   i. Species richness, i.e., the numeric count of ‘present’ species of M_{H}.
   ii. (geometric) Mean of their relative abundances.
b) Basic assumptions:
   i. ψ_{M}_{_{H}} is positively correlated with R_{M}_{_{H}}, or the richness of M_{H} species. Thus, (their correlation) ρ(ψ_{M}_{_{H}}, R_{M}_{_{H}})>0. Remark: Due to the possible large discrepancy between the cardinality (set size) of M_{H} and that of M_{N}, the proportion of ‘present’ M_{H} species is used. As such, R_{M}_{_{H}} in the above assumption is replaced with

$$\frac{{R}_{{M}_{H}}}{\vert {M}_{H} \vert} .$$

### Thus

$$ρ ( {ψ}_{{M}_{H}} , \frac{{R}_{{M}_{H}}}{\vert {M}_{H} \vert} ) > u .$$

ii. ψ_{M}_{_{H}} is positively correlated with <M_{H}>, or the mean abundance of species in M_{H}. Thus, ρ(ψ_{M}_{_{H}}, (M_{H}))>0. Remark: As it is common in microbiome data to have discrepancies between species' relative abundances to span several orders of magnitude, the geometric mean, rather than the arithmetic mean, is more appropriate to represent the mean relative abundance of M_{H} species. More specifically, the Shannon's diversity index, which is a weighted geometric mean (by definition) and commonly applied in ecological contexts, is used. Thus, for simplicity, (M_{H})≈Σ_{jϵI}_{_{MH}}|nⱼln(nⱼ)| is assumed, where I_{M}_{_{H}} is the index set of M_{H}, and nⱼ is the relative abundance of species j in I_{M}_{_{H}}.
c) Overview:
   i. Given the assumptions in b), as well as the non-negativity of

$$\frac{{R}_{{M}_{H}}}{\vert {M}_{H} \vert}$$

and Σ_{jϵI}_{_{MH}}|nⱼln(nⱼ)|, ψ_{M}_{_{H}} is simply formulated as a product of the aforementioned two traits. Thus, let

$${ψ}_{{M}_{H}} = \frac{{R}_{{M}_{H}}}{\vert {M}_{H} \vert} {\sum }_{j ∈ {I}_{{M}_{H}}} \vert {n}_{j} ln ( {n}_{j} ) \vert .$$

ii. Analogously, let

$${ψ}_{{M}_{N}} = \frac{{R}_{{M}_{N}}}{\vert {M}_{N} \vert} {\sum }_{j ∈ {I}_{{M}_{N}}} \vert {n}_{j} ln ( {n}_{j} ) \vert$$

Identifying h_{i,M}_{_{H}}_{,M}_{_{N}}, i.e., ratio of ψ_{M}_{_{H}} to ψ_{M}_{_{N}}, in sample i.
a) Formally, the log-ratio of ψ_{M}_{_{H}} to ψ_{M}_{_{N}} in sample i can be written as

$${h}_{i , {M}_{H} , {M}_{N}} = {log}_{10} ( \frac{\frac{{R}_{{M}_{H}}}{\vert {M}_{H} \vert} {\sum }_{j ∈ {I}_{{M}_{H}}} \vert {n}_{j} ln ( {n}_{j} ) \vert}{\frac{{R}_{{M}_{N}}}{\vert {M}_{N} \vert} {\sum }_{j ∈ {I}_{{M}_{N}}} \vert {n}_{j} ln ( {n}_{j} ) \vert} ) \qquad ( 4 )$$

b) By definition, |M_{H}| and |M_{N}| is the highest richness that can be obtained by M_{H} and M_{N} species, respectively, in a particular microbiome sample. However, the possibility that these maximum values are rarely obtained cannot be ruled out; if so, then consequently, having a larger set size of M_{H} (or M_{N}) can generally result in a lower distribution of

$$\frac{{R}_{{M}_{H}}}{\vert {M}_{H} \vert} ( or \frac{{R}_{{M}_{N}}}{\vert {M}_{N} \vert} ) ,$$

potentially leading to biases in h_{i,M}_{_{H}}_{,M}_{_{N}} when |M_{H}|»|M_{N}| or |M_{H}|«|M_{N}|. Therefore, the upper limits that can be eventually used in replacement of |M_{H}| and |M_{N}| in Equation (4) should reflect more of what is actually observed in real microbiome data, e.g., samples ranked according to the magnitude observed between R_{M}_{_{H}} and R_{M}_{_{N}}. In this regard, the following procedure to find alternative measures for |M_{H}| and |M_{N}| is used:
i. Identify R_{M}_{_{H}} and R_{M}_{_{N}} for all microbiome samples in groups H and N.
ii. Rank-order all samples consecutively by two criteria: First, by all values of R_{M}_{_{N}} in ascending order (from lowest to highest); and then, by all values of R_{M}_{_{H}} in descending order (from highest to lowest). This sorting strategy prioritizes having the highest possible R_{M}_{_{H}} (but with the constraint of having R_{M}_{_{N}}≈0) for the most top-ranked samples; and having the highest possible R_{M}_{_{N}} (but with the constraint of having R_{M}_{_{H}}≈0) for the most bottom-ranked samples.
iii. Let k_{H} be the closest integer to 1% of the number of samples in group H. As H is composed of 2,636 samples, let k_{H} be 26. Analogously, as N is composed of 1,711 samples, let k_{N} be 17.
iv. Denote |M_{H}|′ as the median R_{M}_{_{H}} from the top k_{H} samples, and denote |M_{N}|′ as the median R_{M}_{_{N}} from the bottom k_{N} samples.
v. Replace |M_{H}| and |M_{N}| in Equation (4) with |M_{H}|′ and |M_{N}|′, respectively.
c) In summary, the ratio of ψ_{M}_{_{H}} to ψ_{M}_{_{N}} in gut microbiome sample i can be written as

$${h}_{i , {M}_{H} , {M}_{N}} = {log}_{10} ( \frac{\frac{{R}_{{M}_{H}}}{{\vert {M}_{H} \vert}^{′}} {\sum }_{j ∈ {I}_{{M}_{H}}} \vert {n}_{j} ln ( {n}_{j} ) \vert}{\frac{{R}_{{M}_{N}}}{{\vert {M}_{N} \vert}^{′}} {\sum }_{j ∈ {I}_{{M}_{N}}} \vert {n}_{j} ln ( {n}_{j} ) \vert} ) \qquad ( 5 )$$

Calculating the balanced accuracy of h_{M}_{_{H}}_{,M}_{_{N}}.
a) The relative abundances of species in M_{H} and those in M_{N} for microbiome sample i can be provided as input features for: i) ψ_{M}_{_{H}} and ψ_{M}_{_{N}}, respectively; and ii) h_{i,M}_{_{H}}_{,M}_{_{N}}, which in turn can classify sample i as healthy (i.e., h_{i,M}_{_{H}}_{,M}_{_{N}}>0), non-healthy (i.e., h_{i,M}_{_{H}}_{,M}_{_{N}}<0), or neither (i.e., h_{i,M}_{_{H}}_{,M}_{_{N}}=0).
b) The classification accuracy or predictive performance of h_{i,M}_{_{H}}_{,M}_{_{N}} is found by testing it on all samples in groups H and N, and then by finding the balanced accuracy χ_{M}_{_{H}}_{,M}_{_{N}} defined in Equation (3).

Determining optimal sets M_{H}^{Y} and M_{N}^{Y}.
a) The final, optimal sets of M_{H}^{Y} and M_{N}^{Y} are found by first considering a range of thresholds θ_{f} and θ_{d}. Every pair of θ_{f} and θ_{d} gives different sets of M_{H} and M_{N}, and in turn, different values of balanced accuracy χ_{M}_{_{H}}_{,M}_{_{N}}.
b) The final, optimal sets of M_{H}^{Y} and M_{N}^{Y} (and their corresponding θ_{f}^{Y} and θ_{d}^{Y}) are determined as those that result in the highest balanced accuracy χ_{M}_{_{H}}_{,M}_{_{N}}^{max}.

MetaCyc pathway functional profiling of stool metagenomes. MetaCyc pathway-level relative abundances in each stool metagenome were quantified by the HUManN v2.0 pipeline using default parameters. The EC-filtered UniRef90 gene family database was integrated within the pipeline. Pathways that were unmapped (or unintegrated) were excluded from the analyses.

Designing a classifier based upon Random Forests. A classifier based upon a Random Forests algorithm was designed and curated in Python v3.6.4., while model implementation was performed in the ‘scikit-learn’ Python package v0.23.1.

Stool sample collection and processing. All stool samples from patients with rheumatoid arthritis were obtained following written informed consent. The collection of biospecimens was approved by the Mayo Clinic Institutional Review Board (#14-000616). Stool samples from patients with rheumatoid arthritis were stored in their house-hold freezer (−20° C.) prior to shipment on dry ice to the Medical Genome Facility Research Core at Mayo Clinic (Rochester, Minn.). Once received, the samples were stored at −80° C. until DNA extraction. DNA extraction from stool samples was conducted as follows: Aliquots were created from parent stool samples using a tissue punch, and the resulting child samples were then mixed with reagents from the Qiagen Power Fecal Kit. This included adding 60 uL of reagent C1 and the contents of a power bead tube (garnet beads and power bead solution). These were then vigorously vortexed to bring the sample punch into solution and centrifuged at 18000 G for 15 min. From there, the samples were added into a mixture of magnetic beads using a JANUS liquid handler. The samples were then run through a Chemagic MSM1 according to the manufacturer's protocol. After DNA extraction, paired-end libraries were prepared using 500 ng genomic DNA according to the manufacturer's instructions for the NEB Next Ultra library prep kit (New England BioLabs). The concentration and size distribution of the completed libraries was determined using an Agilent Bioanalyzer DNA 1000 chip (Santa Clara, Calif.) and Qubit fluorometry (Invitrogen, Carlsbad, Calif.). Libraries were sequenced at 23-70 million reads per sample following Illumina's standard protocol using the Illumina cBot and HiSeq 3000/4000 PE Cluster Kit. The flow cells were sequenced as 150×2 paired-end reads on an Illumina HiSeq 4000 using the HiSeq 3000/4000 sequencing kit and HiSeq Control Software HD 3.4.0.38. Base-calling was performed using Illumina's RTA version 2.7.7.

Results

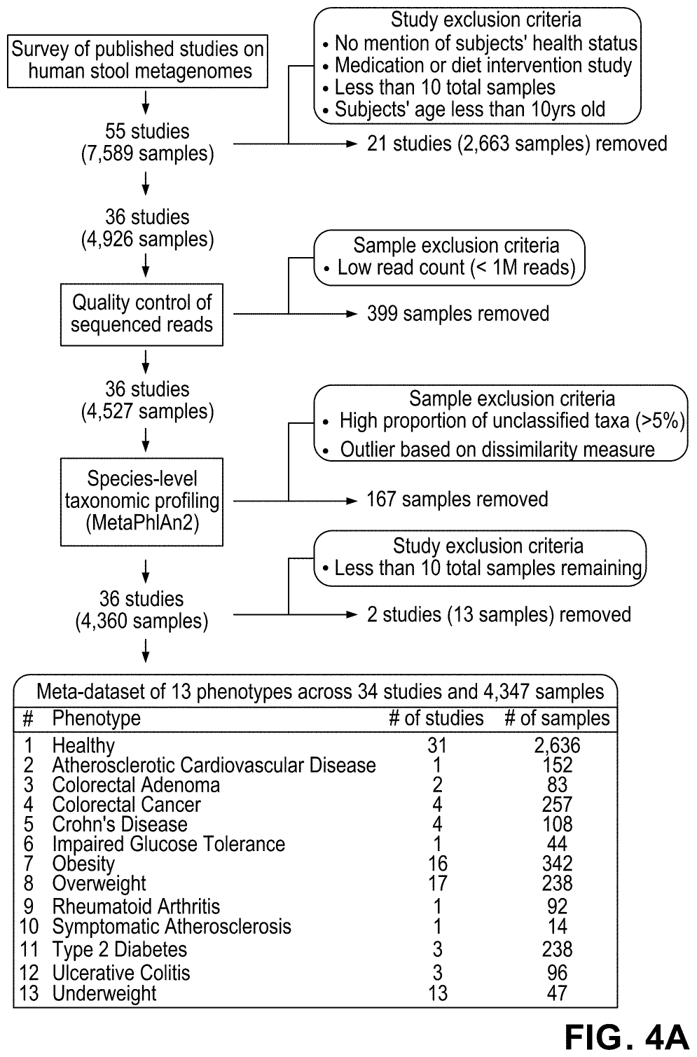

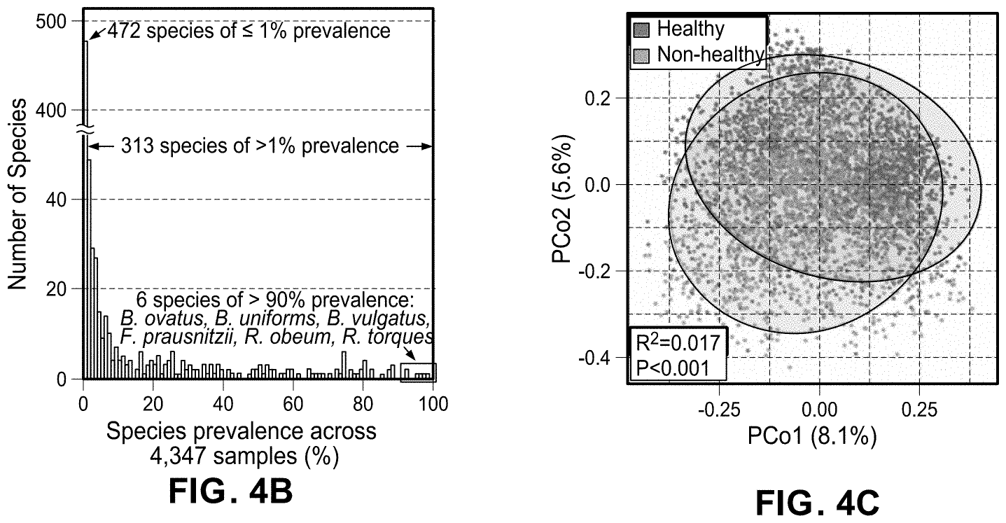

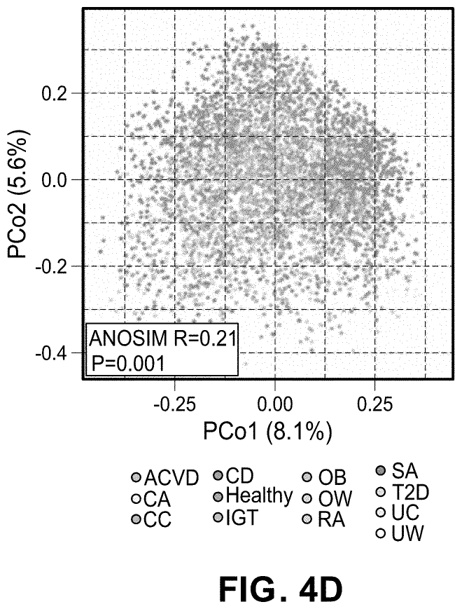

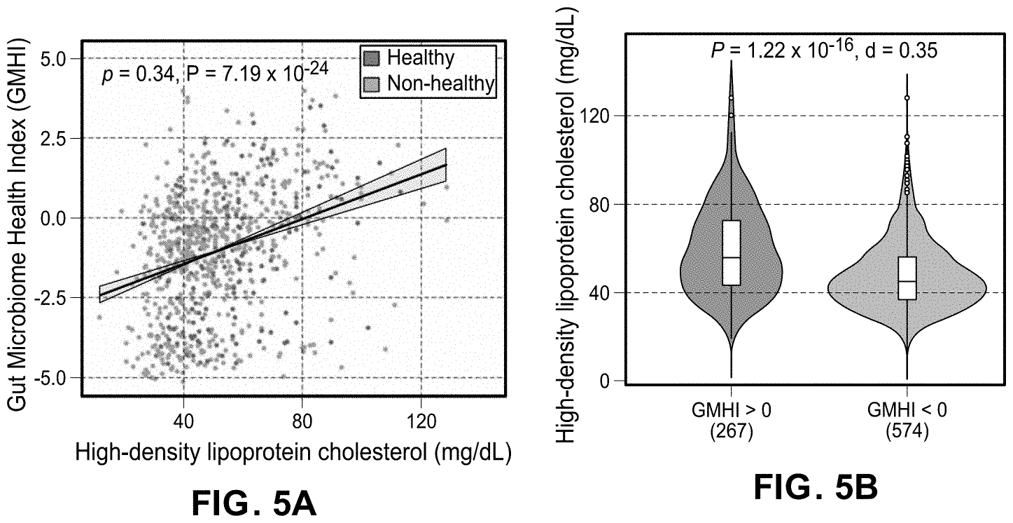

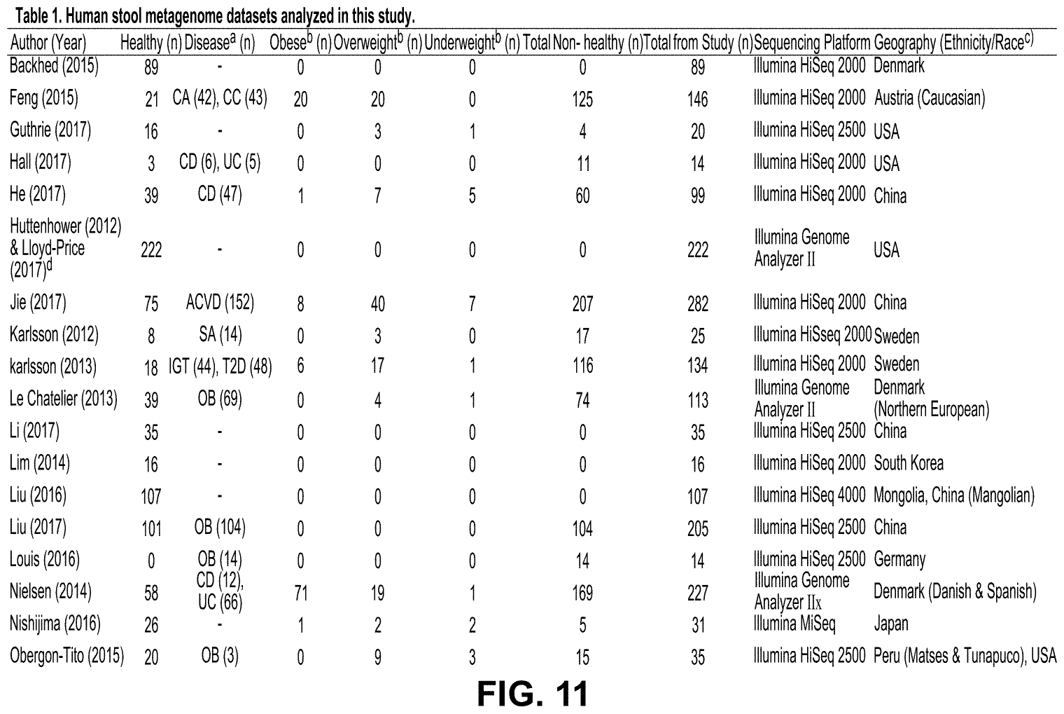

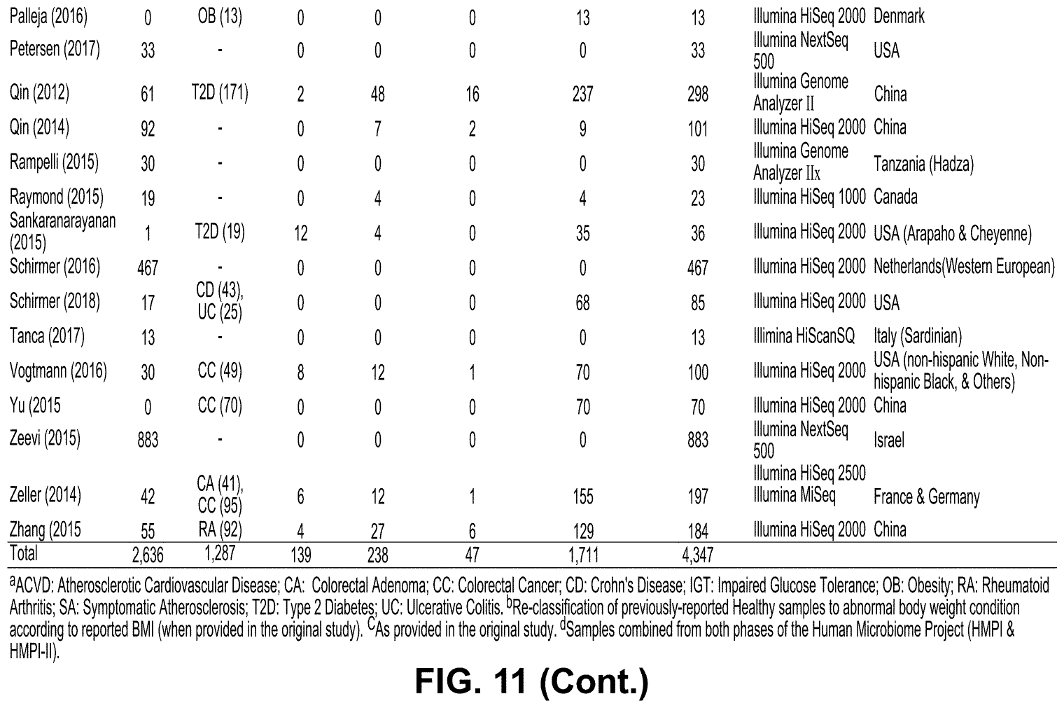

A meta-dataset of integrated human stool metagenomes. An overview of the multi-study integration approach, wherein 4,347 raw shotgun stool metagenomes were acquired (2,636 and 1,711 metagenomes from healthy and non-healthy individuals, respectively) from 34 independent published studies, is depicted in FIG. 4a. In this study, ‘healthy’ subjects were defined as those who were reported as not having any overt disease nor adverse symptoms at the time of the original study; alternatively, ‘non-healthy’ subjects were defined as those who were clinically diagnosed with a disease, or determined to have abnormal bodyweight based on body mass index (BMI). Accordingly, 1,711 stool metagenomes from patients across 12 different disease or abnormal bodyweight conditions were pooled together into a single aggregate non-healthy group. All metagenomes were re-processed uniformly, thereby removing a major non-biological source of variance among different studies. A description of the studies whose human stool metagenomes were collected and processed through the computational pipeline is provided in FIG. 11. In order to eventually identify features of the gut microbiome associated exclusively with health, it is important to be disease-agnostic by considering a broad range of non-healthy phenotypes.

It was chosen to integrate datasets from independent studies for two notable advantages: i) the expansion of sample number could help to amplify the primary biological signal of interest and improve statistical power; and ii) the identified health/disease-associated signatures could encompass a wide range of heterogeneity across different sources and conditions (e.g., host genetics, geography, dietary and lifestyle patterns, age, sex, birth mode, early life exposures, medication history), thereby helping to identify robust findings despite systematic biases from batch effects or other confounding factors.

After downloading, re-processing, and performing quality filtration on all raw metagenomes, species-level taxonomic profiling was carried out using the MetaPhIAn2 pipeline. Of note, the study was mainly conducted upon species-level taxonomy information to obtain as much precise and comprehensive information about the gut microbiome as possible. A total of 1,201 species were detected in at least one metagenome sample; after removing viruses, and species that were rarely observed or of unknown/unclassified identity, 313 species remained for further analysis (FIG. 4b). Interestingly, six species (Bacteroides ovatus, Bacteroides uniformis, Bacteroides vulgatus, Faecalibacterium prausnitzii, Ruminococcus obeum, and Ruminococcus torques) were of high prevalence (i.e., detected in over 90% of all 4,347 samples).

Healthy and non-healthy guts show species-level differences. The overall ecology of the gut microbiome has often been associated with host health. Using species-level relative abundance (i.e., proportion) profiles, the study examined for differences in gut microbial diversity between the healthy and non-healthy groups. First, when using Principal Coordinates Analysis (PCoA) ordination, a significant difference was identified between the distributions of these two groups (PERMANOVA, R²=0.02, P<0.001; FIG. 4c). In the same PCoA plot in which the healthy and twelve non-healthy phenotypes were presented simultaneously (FIG. 4d), only a weak difference was found amongst groups (ANOSIM R=0.21, P=0.001).

Design rationale for a gut microbiome health index. It is envisioned that an especially intuitive way to determine how closely one's microbiome resembles that of a healthy (or non-healthy) population is to quantify the balance between health-associated microbes relative to disease-associated microbes. Therefore, this study proposes an index in the form of a rational equation (and thereby yielding a dimensionless quantity) between two sets of microbial species: those that are more frequently observed in healthy compared to non-healthy groups vs. those that are less frequently observed in healthy compared to non-healthy groups. Next, the compendium of publicly-available datasets is used, which were derived from healthy and non-healthy human subjects, to identify these two sets of species. Finally, with these species, the parameters of a pre-defined formula are tuned, as well as evaluate its classification accuracy. The logical rationale of each major step during the development, demonstration, and validation of the index for predicting general health status (presence/absence of diagnosed disease) from a gut microbiome sample is detailed below.

A prevalence-based strategy to identify health-associated microbes. This study set out to identify distinct microbial species associated with healthy (H) and non-healthy (N) groups. Here, a prevalence-based strategy was used to deal with the sparse nature of microbiome datasets. For this, p_{H,m} and p_{N,m} were determined, or the prevalence of microbial species m in H and N, respectively. (prevalence corresponds to the proportion of samples in a given group wherein m is considered ‘present’, i.e., relative abundance ≥1.0×10⁻⁵.) Next, for comparing the two prevalences in H and N, the following two criteria were applied: prevalence fold-change fₘ^{H,M} and prevalence difference dₘ^{H,N}, defined as

$$\frac{{p}_{H , m}}{{p}_{N , m}}$$

and p_{H,m}-p_{N,m}, respectively. A significant effect-size between the two prevalences is considered to exist if both criteria satisfy (pre-determined) minimum thresholds for prevalence fold-change θ_{f} and prevalence difference θ_{d}. For all detectable microbial species that simultaneously satisfy fₘ^{H,N}≥θ_{f} and dₘ^{H,N}≥θ_{d}, these species observed more frequently in H (than in N) are termed as ‘Health-prevalent’ species M_{H}. Analogously, the study identifies ‘Health-scarce’ species M_{N}, or the species observed less frequently in H (than in N), as those that satisfy fₘ^{N,H}≥θ_{f} and dₘ^{N,H}≥θ_{d}, where fₘ^{N,H} and dₘ^{N,H} is defined as pN,m/pH,m and p_{N,m}-p_{H,m}, respectively. In this regard, the species that are eventually chosen to compose M_{H} and M_{N} are both dependent on θ_{f} and θ_{d}. An important strength of this prevalence-based strategy for identifying microbial associations is that it does not calculate or compare averages of measurements taken from various sources, which is challenging to justify when biological and technical heterogeneity could vary greatly across independent studies. Rather, the present approach compares frequencies of a signal—on a sample-by-sample basis—between two groups, and represents a strategy more applicable to the context of integrating high-throughput data from different studies. It was chosen to simultaneously test two thresholds, rather than one, in order to increase confidence in the robustness of M_{H} and M_{N}, as well as to overcome biases that can occur from using only one type of threshold.

Collective abundances of two sets of microbial taxonomies. Having a strategy to identify microbial species associated with healthy (i.e., Health-prevalent species M_{H}) and non-healthy (i.e., Health-scarce species M_{N}), these two species sets were then coupled with a computational procedure that quantifies the presence/absence of diagnosed disease for any gut microbiome sample. To this end, the following mathematical formula was developed: for species of M_{H} in sample i, their ‘collective abundance’ ψ_{M}_{_{H}}_{,i} is defined as:

$${ψ}_{{M}_{H} , i} = \frac{{R}_{{M}_{H} , i}}{\vert {M}_{H} \vert} {\sum }_{j ∈ {I}_{{M}_{H}}} \vert {n}_{j , i} ln ( {n}_{j , i} ) \vert \qquad ( 1 )$$

where R_{M}_{_{H}}_{,i} is the richness of M_{H} species in sample i, |M_{H}| is the set size of M_{H}, I_{M}_{_{H}} is the index set of M_{H}, and n_{j,i} is the relative abundance of species j in sample i. In brief, ψ_{M}_{_{H}}_{,i} is the product of the i) richness, i.e., the numeric count of ‘present’ taxonomies, of M_{H} species; and ii) the geometric mean of their relative abundances. For the species of M_{N} in the same sample i, their ‘collective abundance’ ψ_{M}_{_{N}}_{,i} can be defined analogously. Next, the collective abundances of species in sets M_{H} and M_{N} in sample i are compared using the ratio of ψ_{M}_{_{H}}_{,i} to ψ_{M}_{_{N}}_{,i} as

$${h}_{i , {M}_{H} , {M}_{N}} = {log}_{10} ( \frac{{ψ}_{{M}_{H} , i}}{{ψ}_{{M}_{N} , i}} ) \qquad ( 2 )$$

where h_{i,M}_{_{H}}_{,M}_{_{N}} denotes the degree to which sample i portrays the collective abundance of M_{H} to that of M_{N}. More specifically, a positive or negative h_{i,M}_{_{H}}_{,M}_{_{N}} suggests that sample i is characterized more by the microbes of M_{H} or M_{N}, respectively; an h_{i,M}_{_{H}}_{,M}_{_{N}} of 0 indicates that there is an equal balance of both species sets.

Determining Health-prevalent and Health-scarce species. The minimum thresholds θ_{f} and θ_{d} for prevalence fold-change and prevalence difference, respectively, are used to control for the number of Health-prevalent species M_{H} and Health-scarce species M_{N}; species that simultaneously satisfy the two types of thresholds are selected to be included in one of either group. Afterwards, M_{H} and M_{N} is provided as input features for ψ_{M}_{_{H}}_{,i} and ψ_{M}_{_{N}}_{,i}, respectively, and for the calculation of h_{i,M}_{_{H}}_{,M}_{_{N}}, which in turn can classify stool metagenome sample i as healthy (i.e., h_{i,M}_{_{H}}_{,M}_{_{N}}>0), non-healthy (i.e., h_{i,M}_{_{H}}_{,M}_{_{N}}<0), or neither (i.e., h_{i,M}_{_{H}}_{,M}_{_{N}}=0). Lastly, h_{i,M}_{_{H}}_{,M}_{_{N}} is tested on all 4,347 stool metagenomes in the meta-dataset to find the balanced accuracy χ_{M}_{_{H}}_{,M}_{_{N}}, i.e., an average of the proportions of healthy and non-healthy samples that were correctly classified, or

$${χ}_{{M}_{H} , {M}_{N}} = \frac{P ( {h}_{i , {M}_{H} , {M}_{N}} > 0 \vert i ∈ H ) + P ( {h}_{i , {M}_{H} , {M}_{N}} < 0 \vert i ∈ N )}{2} \qquad ( 3 )$$

where P(h_{i,M}_{_{H}}_{,M}_{_{N}}>0|l ϵ H) is the proportion of samples in the healthy group (H) whose h_{i,M}_{_{H}}_{,M}_{_{N}}s are positive, and P(h_{i,M}_{_{H}}_{,M}_{_{N}}<0|i ϵ N) is the proportion of samples in the non-healthy group (N) whose h_{i,M}_{_{H}}_{,M}_{_{N}}s are negative.

The final, optimal sets of Health-prevalent and Health-scarce species (and their corresponding prevalence thresholds) were determined as those that result in the highest balanced accuracy χ_{M}_{_{H}}_{,M}_{_{N}}^{max}. This was done accordingly: After systematically testing across a range of two different thresholds (every pair of θ_{f} and θ_{d} gives different sets of M_{H} and M_{N}, and in turn, a different χ_{M}_{_{H}}_{,M}_{_{N}}), it was found that χ_{M}_{_{H}}_{,M}_{_{N}}^{max} to be 69.7% when θ_{f} and θ_{d} were set to 1.4 and 10%, respectively. When applying the same approach for abundance profiles of all other taxonomic ranks, as well as of MetaCyc pathways, the highest accuracies found in these were as follows: Phylum, 42.1%; Class, 60.1%; Order, 62.4%; Family, 67.2%; Genus, 68.2%; and MetaCyc pathway, 59.4%. As evidenced by these results, taxonomic species shows the best classification accuracy. In addition, performing our method in 10-fold cross-validation using species-level abundances resulted in an accuracy of 69.6%, which is nearly identical to the balanced accuracy of 69.7% achieved by testing on the set of samples from which the classifier was derived. Lastly, a sensitivity analysis was shown of how the balanced accuracy χ changes with respect to the species' prevalence thresholds θ_{f} and θ_{d}.

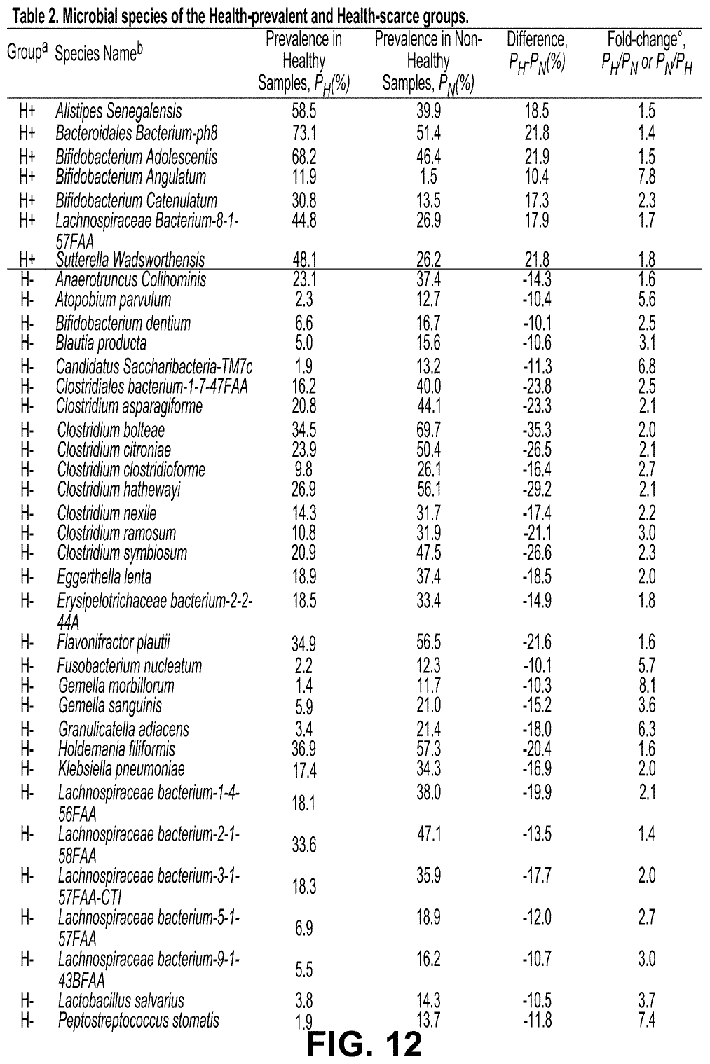

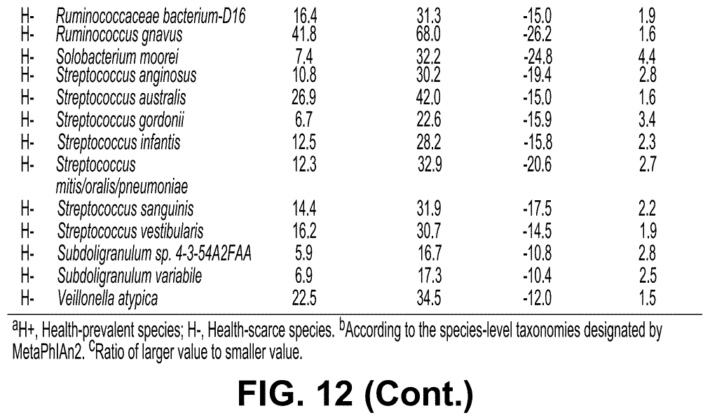

Fifty microbial species were identified that satisfy both of the aforementioned thresholds simultaneously; among these 50 species, 7 and 43 comprise the Health-prevalent and Health-scarce groups, respectively (FIG. 12). Interestingly, the study found higher relative abundance levels of Health-prevalent and Health-scarce species in the healthy and non-healthy group, respectively. Furthermore, the prevalence is shown of these species in case (i.e., non-healthy) and/or control (i.e., healthy) for the 34 published studies upon which the present study's stool metagenome meta-dataset was derived. Despite the heterogeneity and unevenness in prevalences across all studies, it was found that, by and large, Health-prevalent and Health-scarce species were observed more frequently in the control and case samples, respectively.

Henceforth, the ratio h_{i,M}_{_{H}}_{,M}_{_{N}} between these two groups of 7 Health-prevalent and 43 Health-scarce species is referred to as the Gut Microbiome Health Index (GMHI). GMHI is a dimensionless metric designed to simplify the accumulation of Health-prevalent and Health-scarce species observed to be present in a microbiome sample. In practice, GMHI indicates the degree to which a subject's stool metagenome sample portrays microbial taxonomies associated with either healthy or non-healthy.

Analogous to the example mentioned above, a positive or negative GMHI allows the sample to be classified as healthy or non-healthy, respectively; a GMHI of 0 indicates an equal balance of Health-prevalent and Health-scarce species, and thereby classified as neither. Therefore, GMHI is especially favorable in terms of the simplicity of the decision rule and the biological interpretation regarding the two sets of microbes involved in classification. The GMHI metric can be measured on a per sample basis, requires very little parameter-tuning, and foregoes the use of qualitative assessments, e.g., ‘low’ or ‘high’ α-diversity. Furthermore, no significant association was found between library size and GMHI (mixed-effects linear regression, P=0.45), and that, by and large, the distributions of the index for healthy individuals do not vary much between studies.

GMHI is associated with high-density lipoprotein cholesterol. To see whether GMHI can encompass certain physiological features of health, the study looked for statistical associations between GMHI and well-recognized components of physiological wellness from clinical lab tests. More specifically, the study searched for correlations with GMHI and the following, as reported in their original studies: circulating blood concentrations of fasting blood glucose (from 785 subjects), triglycerides (from 915 subjects), total cholesterol (from 521 subjects), low-density lipoprotein cholesterol (LDLC; from 848 subjects), and high-density lipoprotein cholesterol (HDLC; from 841 subjects). Of note, self-reported well-being, treatment regimens, and other questionnaire data were either not provided at all or too sparsely collected to have any practical or statistical significance. When selecting for moderate correlations or better, i.e., |Spearman's ρ|≥0.3 (P<0.001), HDLC was identified as the only feature that was significantly associated with GMHI (ρ=0.34, 95% confidence interval (CI): [0.28, 0.40], P=7.19×10⁻²⁴); in addition, significantly higher abundances of HDLC was identified in subjects with positive GMHI compared to those with negative GMHI (Mann-Whitney U test, P=1.22×10⁻¹⁶). This moderately positive correlation is encouraging for linking GMHI to actual health, as HDLC in the bloodstream is commonly considered as “good” cholesterol, and could be protective against heart attack and stroke, according to the American Heart Association. The study's findings demonstrate the benefit of integrating clinical data with gut microbiome, and also hints at the possibility of GMHI serving as an effective and reliable predictor of cardiovascular health. In contrast, fasting blood glucose (ρ=−0.06, 95% CI: [−0.12, 0.01]), triglycerides (ρ=−0.13, 95% CI: [−0.19, −0.06]), total cholesterol (ρ=0.15, 95% CI: [0.06, 0.23]), LDLC (ρ=0.09, 95% CI: [0.03, 0.16]), and even age (ρ=0.04, 95% CI: [−0.01, 0.08]) were noted to have only weak or no meaningful correlations with GMHI.

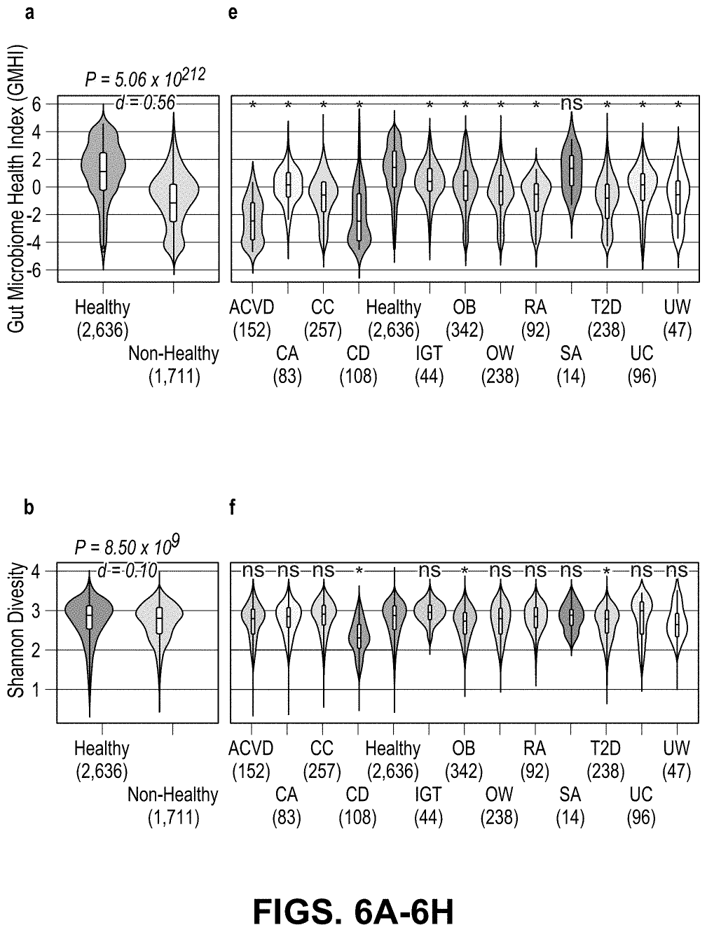

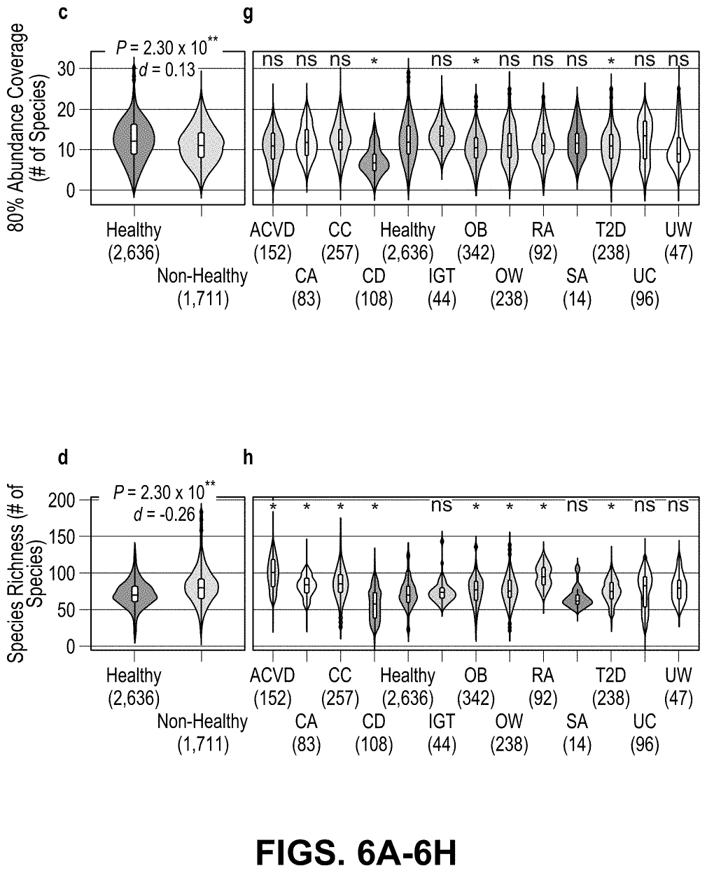

Species-level GMHI stratifies healthy and non-healthy groups. GMHI was calculated for each stool metagenome in our meta-dataset of 4,347 samples to investigate whether the distributions of GMHI differ between healthy and non-healthy groups. It was found that gut microbiomes in healthy have significantly higher GMHIs in comparison to gut microbiomes in non-healthy (Mann-Whitney U test, P=5.06×10⁻²¹²; Cliff's Delta effect-size=0.56; FIG. 6a). (Of note, Cliff's Delta (d) is a non-parametric effect-size measure that quantifies how often one value in one distribution is higher than the values in the second distribution; it is a difference between probabilities, and thus ranges from −1 to +1.) By definition of GMHI, this result reflects the dominant influence of Health-prevalent species over Health-scarce species in the healthy group, and vice versa in the non-healthy group.

Next, to further identify differences between healthy and non-healthy groups, the study examined multiple measures of ecological characteristics that can be defined on a per-sample basis. For α-diversity based on the Shannon index, the study found significantly higher values in healthy than in non-healthy (Mann-Whitney U test, P=8.50×10⁻⁹; Cliff's Delta=0.10; FIG. 6b). The study also found that the minimum number of species to comprise at least 80% of the sample's relative abundance (henceforth called ‘80% abundance coverage’) was significantly higher in healthy compared to non-healthy (Mann-Whitney U test, P=2.30×10⁻¹²; Cliff's Delta=0.13; FIG. 6c). Finally, species richness, which is defined as the observed number of different species, was found to be significantly lower in healthy compared to non-healthy (Mann-Whitney U test, P=2.30×10⁻⁴⁶; Cliff's Delta=−0.26; FIG. 6d).

Finally, the study investigated for differences in GMHI and in these ecological characteristics between healthy and each of the twelve phenotypes of the non-healthy group. At the individual phenotype-level, the healthy group showed significantly higher GMHI levels in all but one (symptomatic atherosclerosis) of the twelve different disease or abnormal bodyweight conditions (Mann-Whitney U test, P<0.001; FIG. 6e). For Shannon diversity and 80% abundance coverage, the study found that only three (Crohn's Disease, Obesity, and Type 2 diabetes) of the twelve non-healthy phenotypes showed statistically significant differences (FIGS. 6f and 6g); both properties were higher in healthy for all three comparisons. For richness, the study found that eight of the twelve non-healthy phenotypes were significantly different compared to healthy (FIG. 6h): seven of these eight were of higher richness, whereas one (Crohn's Disease) was of lower richness. Taken together, the results suggest that: i) healthy and non-healthy gut microbiomes show distinct ecological characteristics; ii) GMHI embodies a gut microbiome signature of wellness that is generalizable against various non-healthy phenotypes; and iii) GMHI can distinguish healthy from non-healthy individuals more reliably than Shannon diversity, 80% abundance coverage, and richness.

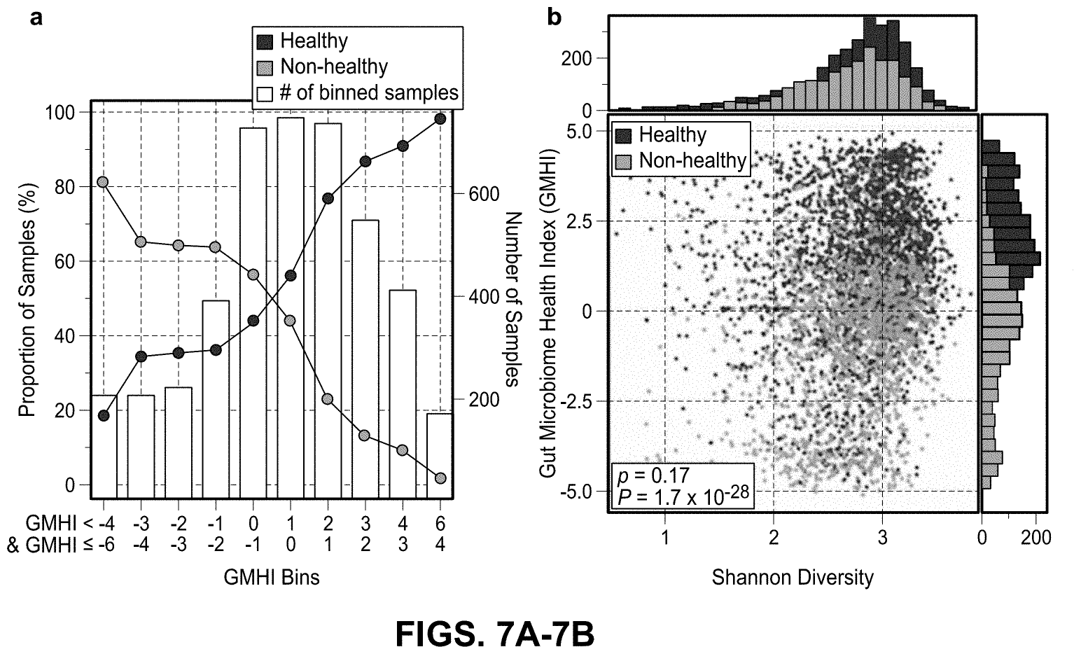

Group proportions and Shannon diversity with respect to GMHI. For increasingly higher (more positive) and lower (more negative) values of GMHI, the study observed an increasing proportion of samples from healthy and non-healthy groups, respectively (FIG. 7a). For example, 98.2% (165 of 168) of metagenome samples with GMHIs higher than 4.0 were from the healthy group; and 81.2% (164 of 202) of metagenome samples with GMHIs lower than −4.0 were of non-healthy origin. In addition, the top 10 to 100 healthy and non-healthy stool metagenome groups (selected based on their GMHIs) clearly clustered apart from each other in PCoA ordination, in stark contrast to the case when all samples were projected simultaneously (FIG. 4c). These observations confirm that very high (or low) collective abundance of Health-prevalent species relative to that of Health-scarce species is strongly connected to being healthy (or non-healthy).

GMHI and Shannon diversity were compared for each sample to examine their overall concordance. As shown in FIG. 7b, GMHI clearly performed much better in stratifying the healthy and non-healthy groups compared to Shannon diversity. A small yet significant relationship was found between our metric and this conventional measure of gut health (Spearman's ρ=0.17, 95% CI: [0.14, 0.19], P=1.66×10⁻²⁸). Additionally, similar results were seen when GMHI was compared with 80% abundance coverage (Spearman's ρ=0.22, 95% CI: [0.19, 0.25], P=8.48×10⁻⁴⁸) and with richness (Spearman's ρ=−0.27, 95% CI: [−0.30, −0.24], P=4.27×10⁻⁷⁴).

Intra-study analyses favor GMHI over other ecology metrics. The study next examined how well GMHI and other features of microbial ecology (i.e., Shannon diversity, 80% abundance coverage, and species richness) could distinguish healthy and non-healthy phenotypes within individual studies. Specifically, in each of the twelve studies (out of 34 total) wherein at least 10 stool metagenome samples from both case (i.e., disease or abnormal bodyweight conditions) and control (i.e., healthy) subjects were available, the study compared GMHI, Shannon diversity, 80% abundance coverage, and species richness between healthy and non-healthy phenotype(s). By focusing on datasets from individual studies one-by-one, this approach not only removes a major source of batch effects, but also provides a good means to investigate the robustness of previously observed trends (when healthy and non-healthy samples were compared against each other in aggregate groups) across multiple, smaller studies.

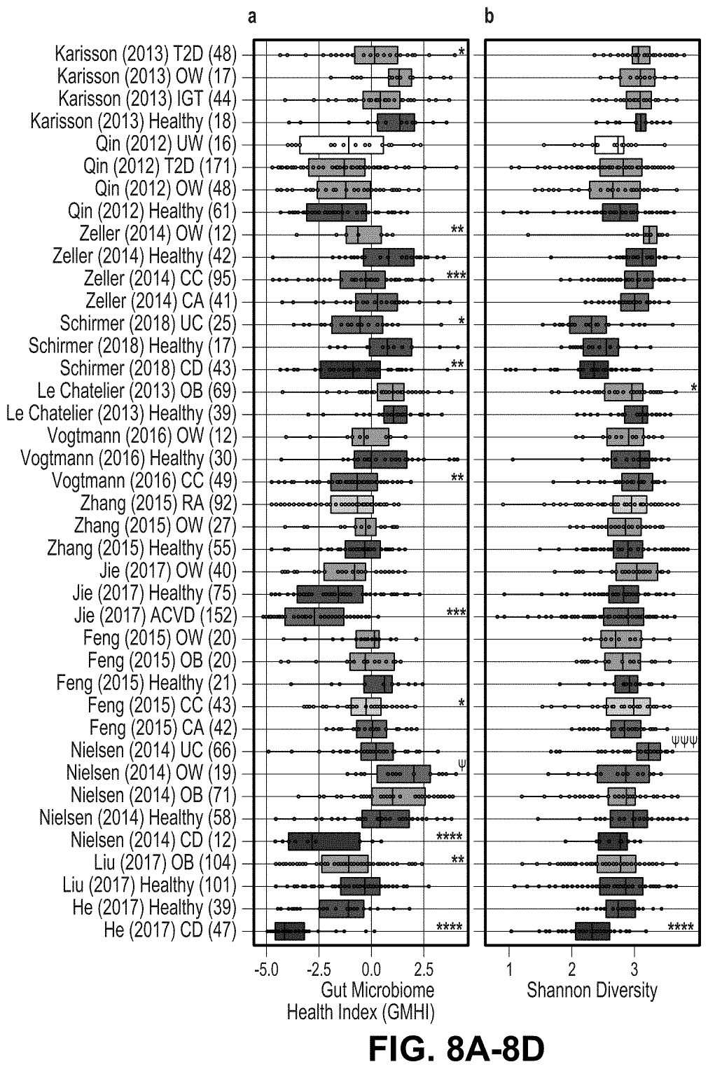

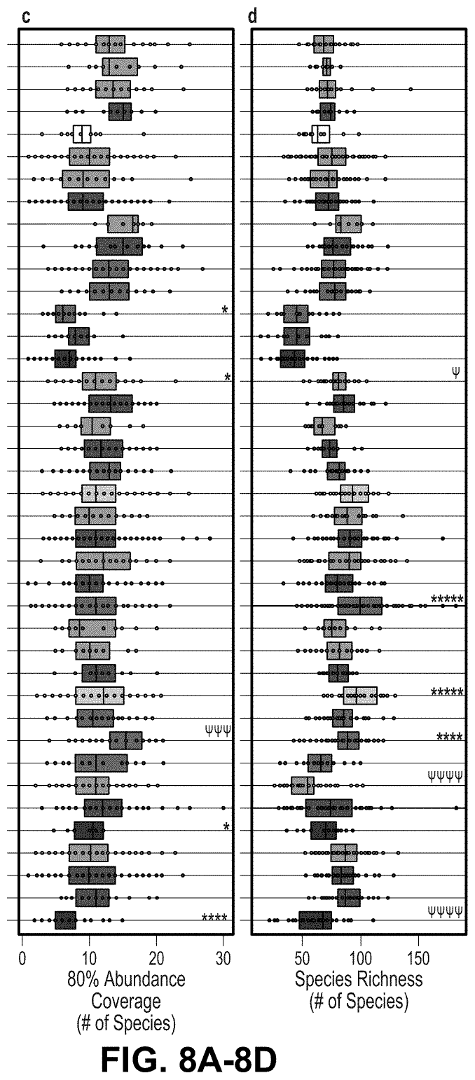

The study found that GMHI in healthy was significantly higher than that in any non-healthy phenotype for eleven out of 28 case-control comparisons (FIG. 8a). For Shannon diversity and 80% abundance coverage, the study found significantly higher values in healthy than in non-healthy phenotypes for two and four case-control comparisons, respectively (FIGS. 8b and 8c). Lastly, the study found species richness in healthy to be significantly lower than that in non-healthy phenotypes for three case-control comparisons (FIG. 8d). Clearly, the performance of GMHI was not perfect (and likewise for other ecological characteristics), as the expected trend from the prior pooled analysis was not replicable for all case-control comparisons within every study; overall though, GMHI strongly outperformed other microbiome ecological characteristics in distinguishing case and control.

Analogous to the analysis above (wherein healthy was compared to each separate non-healthy phenotype within individual studies), the study compared healthy against a general non-healthy phenotype, in which all disease samples were lumped together, when applicable. Importantly, comparisons were still made within individual studies. The study found that there were statistically significant differences in GMHI between cases and controls (Mann-Whitney U test, P<0.05) in six of the twelve studies. In contrast, the study found statistically significant differences in Shannon diversity, 80% abundance coverage, and richness between cases and controls in two, three, and three (of twelve) studies, respectively.

Validation of GMHI reproducibility on independent cohorts. Evaluation of any biomarker or molecular signature on independent patient samples is the gold standard for assessing its robustness. To confirm the reproducibility of our prediction results in stratifying healthy and non-healthy phenotypes, the study leveraged GMHI to predict the health status of 679 individuals whose stool metagenome samples were not part of the original formulation of GMHI. For this, gut microbiome data was used from an additional 8 published studies, which include stool metagenomes from healthy subjects and patients with ankylosing spondylitis (AS), colorectal adenoma (CA), colorectal cancer (CC), Crohn's disease (CD), liver cirrhosis (LC), and non-alcoholic fatty liver disease (NAFLD). In addition, the study utilized extensive biobank of stool collections to gather a set of samples from patients with rheumatoid arthritis (RA). All metagenome samples in this validation dataset were pooled into one of two groups (i.e., healthy or non-healthy), as demonstrated above.

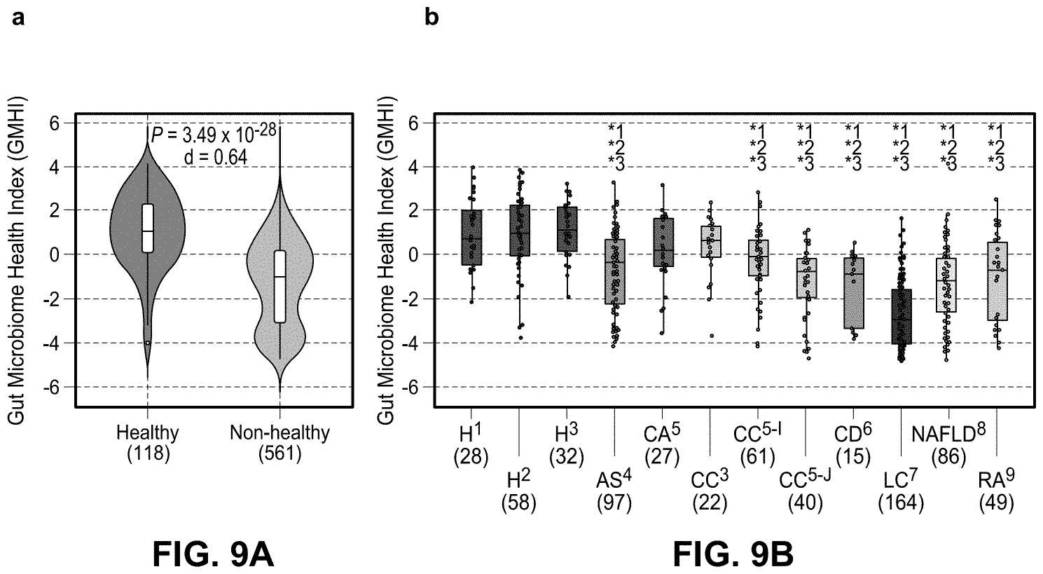

In agreement with the results on the discovery cohort (or training data), GMHIs from stool metagenomes of the healthy validation group (n=118) were significantly higher than those of the non-healthy validation group (n=561) (Mann-Whitney U test, P=3.49×10⁻²⁸; Cliff's Delta=0.64; FIG. 9a). In addition, the balanced accuracy resulted in 73.7%, as the classification accuracy for the healthy and non-healthy validation group was 77.1% (91 of 118) and 70.2% (394 of 561), respectively. Notably, these results were better than the performances on the discovery cohort, wherein balanced accuracy was 69.7%, and accuracy on the healthy and non-healthy group was 75.6% (1,993 of 2,636) and 63.8% (1,092 of 1,711), respectively.

Of note, the study also compared the classification accuracy of GMHI to those of classifiers based upon the Health-prevalent species and Shannon diversity, and to that of a more intricate classification algorithm (Random Forests). In regards to balanced accuracies on the training data, the classifiers based upon Health-prevalent species (χ=66.3%) and Shannon diversity (χ=53.6%) performed comparable to, or much worse than, GMHI (χ=69.7%); furthermore, balanced accuracy on the independent validation dataset for Health-prevalent species and Shannon diversity resulted in 59.3% and 47.0%, respectively. On the other hand, the Random Forests classifier achieved a remarkable accuracy on the training data (χ=98.5%). However, building complex decision rules entails the risk of over-fitting. Surely enough, this nearly perfect accuracy was mostly in part a result of outstanding over-fitting, evidenced by the poor accuracy of 52.3% (balanced accuracy) on the 679 samples of the validation cohort.

To investigate GMHI performances on the validation cohort more closely, the study examined the twelve total sub-cohorts (defined per unique phenotype per individual study) ranging across eight healthy and non-healthy phenotypes from eight additional published studies and one newly sequenced batch. As shown in FIG. 9b, all three healthy sub-cohorts were found to have significantly higher distributions of GMHI than seven (of nine) non-healthy phenotype sub-cohorts (Mann-Whitney U test, P<0.01). The classification accuracies for these three healthy sub-cohorts were 87.5% (28 of 32), 74.1% (43 of 58) and 71.4% (20 of 28); alternatively, the classification accuracies for the non-healthy phenotype sub-cohorts were the following: 94.5% (155 of 164) for Liver cirrhosis (LC); 75.6% (65 of 86) for Non-alcoholic fatty liver disease (NAFLD); 73.3% (11 of 15) for CD; 67.3% (33 of 49) for RA; 55.7% (54 of 97) for ankylosing spondylitis (AS); 37.0% (10 of 27) for CA; and 77.5% (31 of 40), 47.5% (29 of 61), and 27.3% (6 of 22) for three different cohorts of CC. Strikingly, GMHI performed well (>75.0%) in predicting adverse health for LC and NAFLD, although stool metagenomes from patients with liver disease were not part of the original discovery cohort. This finding suggests that GMHI could be applied beyond the original twelve phenotypes (of the non-healthy group) used during the index training process. Overall, the strong reproducibility of GMHI implies that the highly diverse and complex features of gut microbiome dysbiosis implicated in pathogenesis were reasonably well captured during the dataset integration and original formulation of GMHI. Finally, from similar analyses for Shannon diversity, 80% abundance coverage, and species richness on the validation cohort, it was concluded that GMHI is more accurate, robust, and clinically meaningful classifier compared to these other ecological characteristics.

Although various implementations have been described in detail above, other modifications are possible. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

What is claimed is:

1. A method for assessing the gut health of an individual, comprising:
obtaining metagenome data that describes the metagenome for a stool sample of the individual;
determining, based on the metagenome data and for each microbial species of a pre-defined set of microbial species, an indication of presence of the microbial species in the stool sample of the individual;
determining, based on the indications of presence of the microbial species from the pre-defined set of microbial species, a relative presence in the stool sample of microbial species from a first pre-defined subset of the pre-defined set of microbial species to microbial species from a second pre-defined subset of the pre-defined set of microbial species; and
providing an assessment of the gut health of the individual based on the relative presence of microbial species in the stool sample from the first pre-defined subset to microbial species from the second pre-defined subset.

2. The method of claim 1, wherein the method is performed on a computing system having one or more computers in one or more locations.

3. The method of claim 1, wherein the indication of presence of the microbial species comprises a binary indication that the microbial species either has a threshold level of abundance in the stool sample or does not have the threshold level of abundance in the stool sample.

4. The method of claim 1, wherein the indication of presence of the microbial species comprises an indication of a level of abundance of the microbial species in the stool sample.

5. The method of claim 1, further comprising:
obtaining the stool sample from the individual; and
analyzing the stool sample to determine the metagenome data.

6. The method of claim 1, wherein analyzing the stool sample to determine the metagenome data for the stool sample comprises performing at least one of a shotgun sequencing technique on the stool sample, a high-throughput sequencing technique on the stool sample, or a polymerase chain reaction (PCR) technique on the stool sample.

7. The method of claim 1, wherein the microbial species in the pre-defined set of microbial species were selected for inclusion in the pre-defined set based on having been determined to be a statistically significant indicator of gut health such that a presence or lack of presence of the microbial species in studied stool samples was statistically associated with either a healthy gut biome or an unhealthy gut biome.

8. The method of claim 7, wherein the studied stool samples were each classified as being (i) associated with a healthy gut biome if the stool sample was obtained from an individual who was not identified as a having disease and who had a body mass index (BMI) within a normal range, or (ii) associated with an unhealthy gut biome if the stool sample was obtained from an individual who was identified as having disease or who had a BMI outside of the normal range.

9. The method of claim 1, wherein the pre-defined set of microbial species comprises fifty microbial species.

10. The method of claim 1, wherein the first pre-defined subset of microbial species consists of microbial species whose abundance in a stool sample is determined to be a statistically significant indicator of a healthy gut biome.

11. The method of claim 1, wherein the second pre-defined subset of microbial species consists of microbial species whose scarcity in studied stool samples is determined to be a statistically significant indicator of a healthy gut biome.

12. The method of claim 1, wherein the first pre-defined subset of microbial species consists of microbial species that are associated with healthy gut biomes.

13. The method of claim 1, wherein the second pre-defined subset of microbial species consists of microbial species that are associated with unhealthy gut biomes.

14. The method of claim 1, wherein determining the relative presence in the stool sample of microbial species from the first pre-defined subset to microbial species from the second pre-defined subset comprises:
determining a first aggregate indication of presence of microbial species from the first pre-defined subset;
determining a second aggregate indication of presence of microbial species from the second pre-defined subset; and
determining a relationship between the first aggregate indication of presence of microbial species from the first pre-defined subset to the second aggregate indication of presence of microbial species from the second pre-defined subset.

15. The method of claim 14, wherein the relationship comprises a ratio between the first aggregate indication of presence of microbial species from the first pre-defined subset to the second aggregate indication of presence of microbial species from the second pre-defined sub set.

16. The method of claim 15, wherein providing the assessment of the gut health of the individual comprises providing a score indicative of the relative presence in the stool sample of microbial species from the first pre-defined subset to microbial species from the second pre-defined subset.

17. The method of claim 15, further comprising normalizing the score such that a negative score indicates an unhealthy gut biome, a positive score indicates a healthy gut biome, and a zero score indicates a neutral gut biome.

18. The method of claim 15, further comprising:
comparing the score to a threshold value; and
providing an indication of the gut health of the individual based on a result of the comparison of the score to the threshold value.

19. The method of claim 1, further comprising:
generating, based on at least one of the metagenome data or the assessment of the gut health of the individual, a behavioral recommendation that indicates a recommended behavior for the individual to improve gut health; and
providing the behavior recommendation to the individual or another user.

20. (canceled)

21. The method of claim 19, wherein generating the behavioral recommendation comprises accessing, using a computing system, a model that stores data correlating various gut health assessments with corresponding behavioral recommendations.

22. The method of claim 19, wherein providing the behavioral recommendation comprises at least one of presenting the behavioral recommendation on a screen of a computing device or transmitting a representation of the behavioral recommendation over a network.

23. The method of claim 1, wherein providing the assessment of the gut health of the individual comprises presenting the assessment on a screen of a computing device.

24. The method of claim 1, further comprising using a machine-learning model to determine the relative presence in the stool sample of microbial species from the first pre-defined subset to the second pre-defined subset.

25-27. (canceled)
